(19) United States
(12) Patent Application Publication
(10) Pub. No.: US 2024/0124908 A1
(43) Pub. Date: Apr. 18, 2024

(54) METHOD FOR PREPARING (S)-NICOTINE BY REDUCTION

(71) Applicant: Porton Pharma Solutions Ltd., Chongqing (CN)

(72) Inventors: Wenqing LIN, Chongqing (CN); Hongjie ZHENG, Chongqing (CN); Xiaobo LIU, Chongqing (CN); Zecong CHEN, Chongqing (CN); Lingyu LI, Chongqing (CN); Qingjun ZHOU, Chongqing (CN); Songhe WANG, Chongqing (CN); Yongtang YUE, Chongqing (CN); Jicheng HU, Chongqing (CN); Yue ZHANG, Chongqing (CN); Shanshan MIAO, Chongqing (CN)

(21) Appl. No.: 18/522,203

(22) Filed: Nov. 28, 2023

Related U.S. Application Data
(63) Continuation of application No. PCT/CN22/95423, filed on May 27, 2022.

(30) Foreign Application Priority Data
May 29, 2021 (CN) 202110594149.7
May 24, 2022 (CN) 202210575265.9

(51) Int. Cl. C12P 17/12; C07D 401/04

(52) U.S. Cl. CPC C12P 17/12; C07D 401/04; C12Y 101/01047

(57) ABSTRACT

A method for preparing (S)-nicotine by reduction includes conducting a reduction process on an alkene compound as shown in Formula I and/or an iminium cation compound as shown in Formula II, thereby producing (S)-nicotine. The method is simple, safe, reliable, and yields both high purity and high quantities of (S)-nicotine production.

### CROSS-REFERENCE TO RELATED APPLICATIONS

This application is a continuation-in-part of International Patent Application No. PCT/CN2022/095423 with an international filing date of May 27, 2022, designating the United States, now pending, further claims foreign priority benefits to Chinese Patent Application No. 202110594149.7 filed May 29, 2021, and to Chinese Patent Application No. 202210575265.9 filed May 24, 2022. The contents of all of the aforementioned applications, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, MA 02142.

### INCORPORATION-BY-REFERENCE OF SEQUENCE LISTING

This application contains a sequence listing, which has been submitted electronically in XML file and is incorporated herein by reference in its entirety. The XML file, created on Sep. 20, 2023, is named CQBT-00501-UUS.xml, and is 26,302 bytes in size.

### BACKGROUND

The disclosure related to the field of organic synthesis, and more particularly, to a method for preparing (S)-nicotine.

(S)-Nicotine, also known as nicotine, is an alkaloid naturally found in plants belonging to Solanaceae family and constitutes a vital element in tobacco. Typically, tobacco leaves contain 1.5%-3.5% of (S)-nicotine, which is primarily obtained through extraction. While there have been reported chemical synthesis methods for nicotine production, these methods are still in the early stages of development and are more expensive compared to extraction processes. The chemical synthesis methods reported to date include chemical cleavage, asymmetric hydrogenation, the use of chiral auxiliary reagent methods, and so on.

A conventional method for the synthesis of (S)-nicotine involves catalyzing myosmine using a reductase enzyme to produce (S)-nornicotine, followed by the reductive methylation of (S)-nornicotine. The method addresses the issue of cost-effectiveness by employing biocatalysis in the production of (S)-nornicotine, leading to a substantial yield of (S)-nornicotine. Furthermore, in the methylation process, formaldehyde is used as a source of methyl, and formic acid serves as a reducing agent:

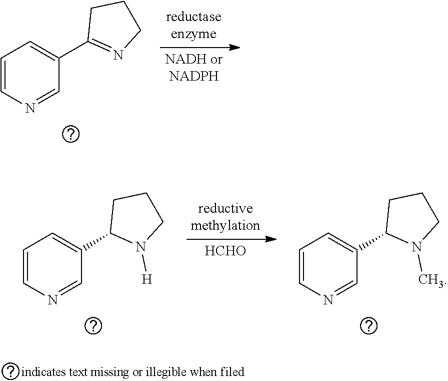 [SMILES: c1cncc(C2=NCCC2)c1 ; c1cncc([C@@H]2CCCN2)c1 ; "HCHO" ; CN1CCC[C@H]1c1cccnc1]

### SUMMARY

The disclosure provides a method for preparing (S)-nicotine by reduction.

The method for preparing (S)-nicotine by reduction comprises:
conducting a reduction process on an alkene compound as shown in Formula I and/or an iminium cation compound as shown in Formula II, thereby producing (S)-nicotine.

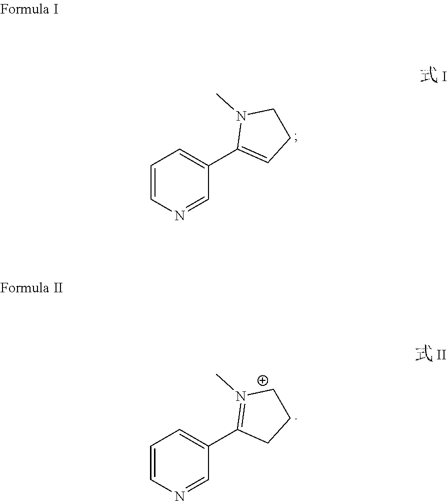

The method prepares (S)-nicotine without the need for methylation by reducing the alkene compound as shown in Formula I and/or the iminium cation compound as shown in Formula II. The method is simple, safe, reliable, and yields high-purity (S)-nicotine.

In a class of this embodiment, the reduction process is performed using a biocatalytic method.

The biocatalytic method comprises:
in a coenzyme cycling system, catalytically reducing the alkene compound as shown in Formula I and/or the iminium cation compound as shown in Formula II by an imine reductase, thereby producing (S)-nicotine.

In a class of this embodiment, the coenzyme cycling system comprises a coenzyme, glucose, and glucose dehydrogenase.

In a class of this embodiment, the coenzyme comprises NADP (Nicotinamide Adenine Dinucleotide Phosphate) salt and/or NAD (Nicotinamide Adenine Dinucleotide) salt, preferably NADP salt.

In a class of this embodiment, the glucose dehydrogenase comprises an amino acid sequence shown in SEQ ID NO: 1.

SEQ ID NO: 1:
MYKDLEGKVVVITGSSTGLGKSMAIRFATEKAKVVVNYRSKEDEANSVL
EEIKKVGGEAIAVKGDVTVESDVINLVQSAIKEFGKLDVMINNAGLENP
VSSHEMSLSDWNKVIDTNLTGAFLGSREAIKYFVENDIKGTVINMSSVH
EKIPWPLFVHYAASKGGMKLMTETLALEYAPKGIRVNNIGPGAINTPIN
AEKFADPEQRADVESMIPMGYIGEPEEIAAVAAWLASSEASYVTGITLF
ADGGMTQYPSFQAGR.

In a class of this embodiment, the imine reductase comprises an amino acid sequence shown in SEQ ID NOs: 2-6, SEQ ID NO: 8, SEQ ID NO: 11, SEQ ID NO: 12, or an amino acid sequence comprising at least 95% identity to the amino acid sequence of SEQ ID NO: 12, preferably the amino acid sequence shown in SEQ ID NOs: 2-4, SEQ ID NO: 12, or the amino acid sequence comprising at least 95% identity to the amino acid sequence of SEQ ID NO: 12.

SEQ ID NO: 2:
MRHLSVIGLGAMGSALATTLLKAGHPVTVWNRSAAKAAPLQALGAT
LAPSVGEAIAASDITLVCVDNYAVSQQLLDEASDAVAGKLLVQLSTGSP
QGARSLESWCHTRGARYLDGAILCFPDQIGTTDASIICSGASTAFSEAE
PVLRLLAPPLDHVAEAVGAAAAQDCAVAAYFAGGLLGALHGALICEVEG
LPVAKVCAQFSELSPILGGDVAHLGKTLASGDFDHPYASLKTWSAAISR
LAGHATDAGIDSRFPRFAADLFEEGVAQGFGQQEVSALIKVLRARNGAA
Q.
SEQ ID NO: 3:
MRHLSVIGLGAMGSALATTLLKAGHPVTVWNRSAAKAAPLQALGAT
LAPSVGAAIAASDITLVCVDNYAVSQLLLDEASDAVAGKLLVQLSTGSP
QGARALESWSHARGARYLDGAILCFPAQIGTSDASIICSGASAAFSEAE
PVLSLLAPTLDHVAEAVGAAAAQDCAVAAYFAGGLLGALHGALICEAEG
LPVAKVCAQFSELSPILGGDVAHLGKTLASGDFDHPYASLKTWSAAISR
LAGHATDAGIDSRFPRFAADLFEEGVAQGFGQQEVSALIKVLRARNGAA
Q.
SEQ ID NO: 4:
MRHLSVIGLGAMGSALATTLIKGGHPVTVWNRSAAKAAPLQALGAT
LAPSVGAAIAASDITLVCVDNYAVSQQLLDEARDAVAGKLLVQLSTGSP
QGARALESWSHARGARYLDGAILCFPDQIGTSDASIICSGASAAYFAGG
LLGALHGALICEAEGLPVAKVCAQFSELSPILGGDVAHLGKTLASGDFD
HPYASLKTWSAAISRLAGHATDAGIDSRFPRFAADLFEEGVAQGFGQQE
VSALIKVLRARNGAAL.
SEQ ID NO: 5:
MRPISVIGLGAMGSALATTLLKAGHPVTVWNRSAAKATPLIALGAIL
APSVSEAIAAGDITLICVDNYAVSQQLLDEASNAVTGKLVVQLSTGSPL
GARTLESWCHARGACYLDGAILCFPDQIGTTDASIICSGANAAFREAEP
VLRLLAPTLEHVAEAVGAAAAQDCAVAAYFAGGLLGALHGALICEAEGL
PVAKVCAQFSELSPILGGDVAHLGKTLASGDFDHPYASLKTWSAAISRL
TDHAADAGIDNSFPRFAADLFEEGVEQGLGQQEVSALIKVLRARNGAA
Q.
SEQ ID NO: 6:
MRHLSVIGLGAMGSALATTLIKAGHPVTVWNRSAAKSAPLQALGAT
LAPSVGAAIAASDITLVCVDNYAVSQQLLDEASDAVAGKQLVQLSTGSP
QGARALESWSHARGARYLDGAILCFPDQIGTSDASIICSGANTAFSDAE
PVLRLLAPTLDHVAEAVGAAAAQDCAVAAYFAGGLLGALHGALICEAEG
LPVTKVCAQFSELSPILGGDVAHLGKTLASGDFDHPYASLKTWSAAISR
LAGHATDAGIDSRFPRFAADLFEEGVAQGFGQQEVSALIKVLRARNGAA
Q.
SEQ ID NO: 7:
MGTALVEAFLAGGHATTVWNRTPGKADGVVARGAVVAETVAEAVAA
SPLVVVCLWDDAVVRDVLHPVADALAGRVVVNLTNGTPAQAREMAAWAA
EHGVEYVDGGIMAIPPGIGTEHAFVLYSGAEAAFEAHREVLERLGAAKY
LGADAGLAALFDLALLSGMYGTFAGLWHSLAMVRTENVSAAEFVPMLGP
WMQAMIGGNLDRLAHQLDTGDYGHEVVSNLAMQAAAFPNIVQASLDQGI
RPDLMAPIQRLMDQAVAAGHGAEDVAVVVDLLKN.
SEQ ID NO: 8:
MKPTLTVIGAGRMGSALIKAFLQSGYTTTVWNRTKAKSEPLAKLGA
HLADTVRDAVKRSDIIVVNVLDYDTSDQLLRQDEVTRELRGKLLVQLTS
GSPALAREQETWARQHGIDYLDGAIMATPDFIGQAECALLYSGSAALFE
KHRAVLNVLGGATSHVGEDVGHASALDSALLFQMWGTLFGTLQALAISR
AEGIPLEKTTAFIKLTEPVTQGAVADVLTRVQQNRLTADAQTLASLEAH
NVAFQHLLALCEERNIHRGVADAMYSVIREAVKAGHGKDDFAILTRFL
K.
SEQ ID NO: 9:
MVSSPYLNVTAYPKVRNLPWPVPGPIRVASQILELRPMTTIGFLGAGR
MGSALVKSLLEAGHSVHVWNRTAEKAQALADFGAVPEPSAERAAGPAEI
VIVNLLDYEASDAELRKPDVAEALKGKLLVQLTSGSPKTARETGRWAGD
HGIAYLDGAIMATPNFIGGAETVILYSGSKTHFEKHEGLFKALGGKSAF
VGEDFGTASALDSALLSQMWGTLFGTLQALAVCRAEGIEHDVYAGFLMS
AQPMIDGAQQDLMERIRDGRDLADAQTLATVAVHNVAFHHLRDLIADRD
LNPAFGDALGSLLETALRNDHQDDDFAVLARFMGAK.
SEQ ID NO: 10:
MTDLGKSAVTVLGLGAMGTALAEALLAAGHPTTVWNRSPARTAGPA
QRGAAVAAATAEAIAASRLIVVCLLDHTSVHAVLDGQELTGRIVVNLTS
GTPGQARELDARVAERGGDHLDGAVLAVPSMIGTPDASVLYSGSRGAFD
THRPVLEVFGAADYVGADPGAASLQDAALLSAMYGQVAGVLHAFALVRS
AGVTATEFLPRLVGWLTAMGGFPADAARRIDARAYADDVDAALTMQVTA
VRNLVRAAREQGVSAELIAPLVPVMQRRIDDGDGGDDLAALVEVITAEE
VA.
SEQ ID NO: 11:
MTDKPPVTVLGLGAMGTALARTLLNAGYPTTVWNRTASKTAPLTEL
GAHAADSPADAIARGELVLACLLDYDSVHQTLAGTGDALRGKAFVNLIN
GTPEQARALAGKLDTAYLDGGIMAVPPMIGSPGAFLFYSGEIAVFEQYR
PVLESFGEAIEVGTDPGLAALHDLALLSAMYGMFGGVLQAFALTGSAGV
SAASLAPLLHRWLDGMSGFIAQSAAQLDSGDFATGVVSNLAMQDTGFAN
LFRAAKEQGISTGQLEPLGALIRRRVEDGHGAEDLAGIVEYLKIGANA.
SEQ ID NO: 12:
MRHLSVIGLGAMGSALATTLLKAGHPVTVWNRSAAKAAPLQALGAT
LAPSVGAAIAASDITLVCVDNYAVSQLLLDEASDAVAGKLLVQLSTGSP
QGARALESWSHARGARYLDGAILCFPAQIGTSDASIICSGASAAFSEAE
PVLSLLAPTLDHVAEAVGAAAAQDCAVAAYFAGGLLGALHGALICEAEG
LPVAKVCAQFSELSPILGGDVAHLGKTLASGDFDHPYASLKTWSAAISR
LAGHATDAGIDSRFPRFAADLFEEGVAQGFGQQEVSALIKVLRARNGAA
Q.

One skilled in the art, employing conventional methods, introduces mutations into one or more amino acids of the amino acid sequence as shown in SEQ ID NOs: 2-12, thereby obtaining imine reductase mutants. The imine reductase mutants are screened for their ability to catalyze the substrates essential for producing (S)-nicotine, with a conversion rate and an enantiomeric excess (ee) value meeting the operational standards established for the imine reductase.

In a class of this embodiment, the amino acid sequence, with at least 95% identity to SEQ ID NO: 12, exhibits mutations compared to the amino acid sequence of SEQ ID NO: 12 in the amino acid residues at the following positions: L73, S148, V171, A172, or a combination thereof.

In a class of this embodiment, the amino acid residue at position 73 is mutated from L to Q or V, the amino acid residue at position 148 is mutated from S to R, the amino acid residue at position 171 is mutated from V to Y, N, A, or S, and the amino acid residue at position 172 is mutated from A to V or F.

In a class of this embodiment, the amino acid sequence, with at least 95% identity to SEQ ID NO: 12, further exhibit mutations compared to the amino acid sequence of SEQ ID NO: 12 in the amino acid residues at the following positions: A57, A176, Y230, S241, or a combination thereof.

In a class of this embodiment, the amino acid residue at position 57 is mutated from A to R, the amino acid residue at position 176 is mutated from A to G, the amino acid residue at position 230 is mutated from Y to G, A, or T, and the amino acid residue at position 241 is mutated from S to G or A.

In a class of this embodiment, the amino acid sequence, with at least 95% identity to SEQ ID NO: 12, is represented by any one of the amino acid sequences as shown in SEQ ID NOs: 13-22.

SEQ ID NO: 13:
MRHLSVIGLGAMGSALATTLLKAGHPVTVWNRSAAKAAPLQALGAT
LAPSVGAAIAASDITLVCVDNYAVSQVLLDEASDAVAGKLLVQLSTGSP
QGARALESWSHARGARYLDGAILCFPAQIGTSDASIICSGASAAFSEAE
PVLSLLAPTLDHVAEAVGAAAAQDCAAAAYFAGGLLGALHGALICEAEG
LPVAKVCAQFSELSPILGGDVAHLGKTLASGDFDHPYASLKTWSAAISR
LAGHATDAGIDSRFPRFAADLFEEGVAQGFGQQEVSALIKVLRARNGAA
Q.
SEQ ID NO: 14:
MRHLSVIGLGAMGSALATTLLKAGHPVTVWNRSAAKAAPLQALGAT
LAPSVGAAIAASDITLVCVDNYAVSQQLLDEASDAVAGKLLVQLSTGSP
QGARALESWSHARGARYLDGAILCFPAQIGTSDASIICSGASAAFSEAE
PVLRLLAPTLDHVAEAVGAAAAQDCAVAAYFAGGLLGALHGALICEAEG
LPVAKVCAQFSELSPILGGDVAHLGKTLASGDFDHPYASLKTWSAAISR
LAGHATDAGIDSRFPRFAADLFEEGVAQGFGQQEVSALIKVLRARNGAA
Q.
SEQ ID NO: 15:
MRHLSVIGLGAMGSALATTLLKAGHPVTVWNRSAAKAAPLQALGAT
LAPSVGAAIAASDITLVCVDNYAVSQQLLDEASDAVAGKLLVQLSTGSP
QGARALESWSHARGARYLDGAILCFPAQIGTSDASIICSGASAAFSEAE
PVLRLLAPTLDHVAEAVGAAAAQDCAVAAYFAGGLLGALHGALICEAEG
LPVAKVCAQFSELSPILGGDVAHLGKTLASGDFDHPGASLKTWSAAISR
LAGHATDAGIDSRFPRFAADLFEEGVAQGFGQQEVSALIKVLRARNGAA
Q.
SEQ ID NO: 16:
MRHLSVIGLGAMGSALATTLLKAGHPVTVWNRSAAKAAPLQALGAT
LAPSVGAAIARSDITLVCVDNYAVSQLLLDEASDAVAGKLLVQLSTGSP
QGARALESWSHARGARYLDGAILCFPAQIGTSDASIICSGASAAFSEAE
PVLSLLAPTLDHVAEAVGAAAAQDCAAVAYFGGGLLGALHGALICEAEG
LPVAKVCAQFSELSPILGGDVAHLGKTLASGDFDHPTASLKTWSAAIGR
LAGHATDAGIDSRFPRFAADLFEEGVAQGFGQQEVSALIKVLRARNGAA
Q.
SEQ ID NO: 17:
MRHLSVIGLGAMGSALATTLLKAGHPVTVWNRSAAKAAPLQALGAT
LAPSVGAAIARSDITLVCVDNYAVSQLLLDEASDAVAGKLLVQLSTGSP
QGARALESWSHARGARYLDGAILCFPAQIGTSDASIICSGASAAFSEAE
PVLSLLAPTLDHVAEAVGAAAAQDCAAVAYFGGGLLGALHGALICEAEG
LPVAKVCAQFSELSPILGGDVAHLGKTLASGDFDHPYASLKTWSAAIGR
LAGHATDAGIDSRFPRFAADLFEEGVAQGFGQQEVSALIKVLRARNGAA
Q.
SEQ ID NO: 18:
MRHLSVIGLGAMGSALATTLLKAGHPVTVWNRSAAKAAPLQALGAT
LAPSVGAAIARSDITLVCVDNYAVSQLLLDEASDAVAGKLLVQLSTGSP
QGARALESWSHARGARYLDGAILCFPAQIGTSDASIICSGASAAFSEAE
PVLSLLAPTLDHVAEAVGAAAAQDCAAAAYFGGGLLGALHGALICEAEG
LPVAKVCAQFSELSPILGGDVAHLGKTLASGDFDHPYASLKTWSAAIGR
LAGHATDAGIDSRFPRFAADLFEEGVAQGFGQQEVSALIKVLRARNGAA
Q.
SEQ ID NO: 19:
MRHLSVIGLGAMGSALATTLLKAGHPVTVWNRSAAKAAPLQALGAT
LAPSVGAAIARSDITLVCVDNYAVSQLLLDEASDAVAGKLLVQLSTGSP
QGARALESWSHARGARYLDGAILCFPAQIGTSDASIICSGASAAFSEAE
PVLSLLAPTLDHVAEAVGAAAAQDCAVVAYFGGGLLGALHGALICEAEG
LPVAKVCAQFSELSPILGGDVAHLGKTLASGDFDHPYASLKTWSAAIGR
LAGHATDAGIDSRFPRFAADLFEEGVAQGFGQQEVSALIKVLRARNGAA
Q.
SEQ ID NO: 20:
MRHLSVIGLGAMGSALATTLLKAGHPVTVWNRSAAKAAPLQALGAT
LAPSVGAAIARSDITLVCVDNYAVSQQLLDEASDAVAGKLLVQLSTGSP
QGARALESWSHARGARYLDGAILCFPAQIGTSDASIICSGASAAFSEAE
PVLRLLAPTLDHVAEAVGAAAAQDCAVAAYFGGGLLGALHGALICEAEG
LPVAKVCAQFSELSPILGGDVAHLGKTLASGDFDHPYASLKTWSAAISR
LAGHATDAGIDSRFPRFAADLFEEGVAQGFGQQEVSALIKVLRARNGAA
Q.
SEQ ID NO: 21:
MRHLSVIGLGAMGSALATTLLKAGHPVTVWNRSAAKAAPLQALGAT
LAPSVGAAIARSDITLVCVDNYAVSQQLLDEASDAVAGKLLVQLSTGSP
QGARALESWSHARGARYLDGAILCFPAQIGTSDASIICSGASAAFSEAE
PVLRLLAPTLDHVAEAVGAAAAQDCAVAAYFGGGLLGALHGALICEAEG
LPVAKVCAQFSELSPILGGDVAHLGKTLASGDFDHPYASLKTWSAAIAR
LAGHATDAGIDSRFPRFAADLFEEGVAQGFGQQEVSALIKVLRARNGAA
Q.
SEQ ID NO: 22:
MRHLSVIGLGAMGSALATTLLKAGHPVTVWNRSAAKAAPLQALGAT
LAPSVGAAIARSDITLVCVDNYAVSQQLLDEASDAVAGKLLVQLSTGSP
QGARALESWSHARGARYLDGAILCFPAQIGTSDASIICSGASAAFSEAE
PVLRLLAPTLDHVAEAVGAAAAQDCAVAAYFGGGLLGALHGALICEAEG
LPVAKVCAQFSELSPILGGDVAHLGKTLASGDFDHPTASLKTWSAAISR
LAGHATDAGIDSRFPRFAADLFEEGVAQGFGQQEVSALIKVLRARNGAA
Q.

One skilled in the art, employing conventional methods, obtains the imine reductase mutants with any combination of the mutation points. The imine reductase mutants are screened to determine their ability to catalyze the substrates essential for producing (S)-nicotine, with a conversion rate exceeding 99% and an ee value exceeding 99%.

In a class of this embodiment, the reduction process is carried out within a temperature range of 15 to 45° C., such as 15° C., 20° C., 25° C., 30° C., 35° C., 40° C., 45° C.; other specific temperature values within the range can also be chosen, and are not detailed herein.

In a class of this embodiment, the reduction process is conducted in a buffer solution; and the buffer solution comprises phosphate buffer, Tris-HCl buffer, or TEA-HCl buffer.

In a class of this embodiment, the reduction process is carried out within a pH range of 6.0 to 8.0, such as pH 6.0, pH 6.2, pH 6.5, pH 6.8, pH 7.0, pH 7.2, pH 7.5, pH 7.8, pH 8.0, and other specific pH values within the range can also be chosen, and are not detailed herein.

In a class of this embodiment, the reduction process is performed using a chiral metal catalyst, and a specific method comprises:
in a hydrogen-rich atmosphere, catalytically reducing the alkene compound as shown in Formula I and/or the iminium cation compound as shown in Formula II by the chiral metal catalyst, thereby yielding (S)-nicotine.

In a class of this embodiment, the chiral metal catalyst comprises a chiral iridium catalyst, a chiral ruthenium catalyst, or a chiral rhodium catalyst, and more preferably, the chiral iridium catalyst.

In a class of this embodiment, a ligand is added to the reduction process.

The ligand is at least one selected from a group comprising a plurality of ligands listed in Table 1, preferably (R,R)-f-SpiroPhos and (S,S)-Ph-BPE.

TABLE 1
(S)-(+)-1-((R)-2-(Diphenylphosphino)ferrocenyl)ethyldi-t-butylphosphine
(S)-(+)-Neomenthyldiphenylphosphine
abbreviated as (S)-NMDPP
(R)-(+)-2,2′-Bis(diphenylphosphino)-1,1′-binaphthyl
abbreviated as (R)-(+)-BINAP
(R,R)-(2-(4′-i-Propyloxazolin-2′-yl)ferrocenyl)diphenylphosphine
(R)-(−)-1-((S)-2-
(Diphenylphosphino)ferrocenyl)ethyldicyclohexylphosphineethanoladduct
abbreviated as (R)-(S)-JOSIPHOS
(R)-(−)-1-((S)-2-(Dicyclohexylphosphino)ferrocenyl)ethyldi-t-
butylphosphine
abbreviated as (R)-(S)-Cy2PF-P(tBu)2
(R)-(−)-1-((S)-2-(Diphenylphosphino)ferrocenyl)ethyldi-t-butylphosphine
abbreviated as (R)-(S)-PPF-P(tBu)2
(+)-1,2-Bis((2S,5S)-2,5-diphenylphospholano)ethane
abbreviated as (S,S)-Ph-BPE
1,2-Bis((2S,5S)-2,5-Methylphospholano)ethane
abbreviated as (S,S)-Me-BPE
(4S,5S)-2,2-Dimethyl-4,5-bis(diphenylphosphinomethyl)-1,3-dioxolane
abbreviated as (S,S)-DIOP
(S)-(+)-(3,5-Dioxa-4-phospha-cyclohepta(2,1-a;3,4-a′)dinaphthalen-4-
yl)dimethylamine
abbreviated as (S)-MONOPHOS
N,N′-(1S,2S)-1,2-Cyclohexanediylbis(2-(diphenylphosphino)benzamide)
abbreviated as (S,S)-DACH-Phenyl Trost Ligand
(R)-(+)-5,5′-Bis(diphenylphosphino)-4,4′-bi-1,3-benzodioxole
abbreviated as (R)-SEGPHOS
(S)-(−)-(6,6′-Dimethoxybiphenyl-2,2′-diyl)bis(diphenylphosphine)
abbreviated as (S)-MeO-BIPHEP
(1S,2S)-(+)-N-(4-toluenesulfony1)-1,2-diphenylethylenediamine
abbreviated as (S,S)-TsDPEN
CAS: 1629646-88-3
abbreviated as (R,R)-f-SpiroPhos
1,2-Bis((2R,5R)-2,5-diethylphospholano)benzene
abbreviated as (R,R)-Et-Duphos

In a class of this embodiment, the reduction process is carried out within a temperature range of 50 to 100° C., such as 50° C., 55° C., 60° C., 65° C., 70° C., 75° C., 80° C., 85° C., 90° C., 95° C., 100° C., other specific temperature values within the range can also be chosen, but are not detailed herein.

In a class of this embodiment, during the reduction process, the pressure of the hydrogen gas is maintained at a range of 1.0 and 6.0 MPa, such as 1.0 MPa, 2.0 MPa, 3.0 MPa, 4.0 MPa, 5.0 MPa, 6.0 MPa, and other specific pressure values within the range can also be chosen, but are not detailed herein.

In a class of this embodiment, the reduction process is carried out within a pH range of 4.0 to 13.0, such as pH 4.0, 5.0, 6.0, pH 6.2, pH 6.5, pH 6.8, pH 7.0, pH 7.2, pH 7.5, pH 7.8, pH 8.0, pH 10.0, pH 12.0, pH 13.0, and other specific pH values within the range can also be chosen, but are not detailed herein.

The alkene compound as shown in Formula I or the iminium cation compound as shown in Formula II are prepared by desalination and/or cyclization of a compound C as shown in Formula III, or a salt thereof.

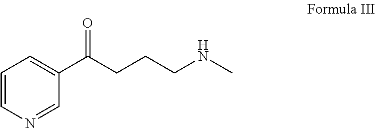

In a class of this embodiment, the salt of the compound C comprises hydrochloride, dihydrochloride, hydrobromide, dihydrobromide, sulfate, or bisulfate.

The compound C or the salt thereof undergo desalination and/or cyclization in the presence of an inorganic base and/or an organic base, thereby yielding the alkene compound as shown in Formula I or the iminium cation compound as shown in Formula II.

A method for synthesizing the salt of the compound C, and the method comprises:
mixing a compound A with N-methylpyrrolidone and an organic base to yield a compound D; and mixing the compound D with an acid to yield the salt of the compound C; the reaction equations are as follows:

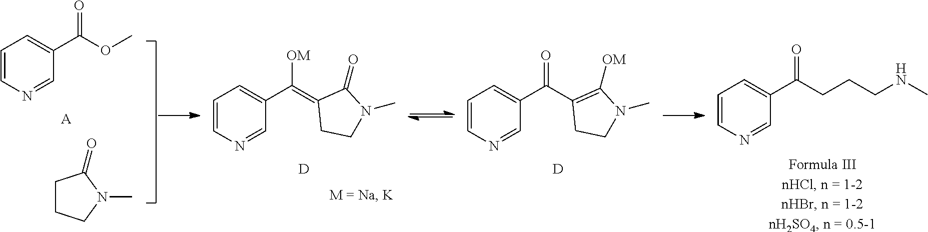

In a class of this embodiment, a method for synthesizing the salt of the compound C, and the method comprises:
mixing the compound A with N-methylpyrrolidone and an organic base to form a resultant product; neutralizing the resultant product with an acid to reach a pH of 7-8, thereby yielding a compound B; and mixing the compound B with an acid to produce the salt of the compound C; the reaction equations are as follows:

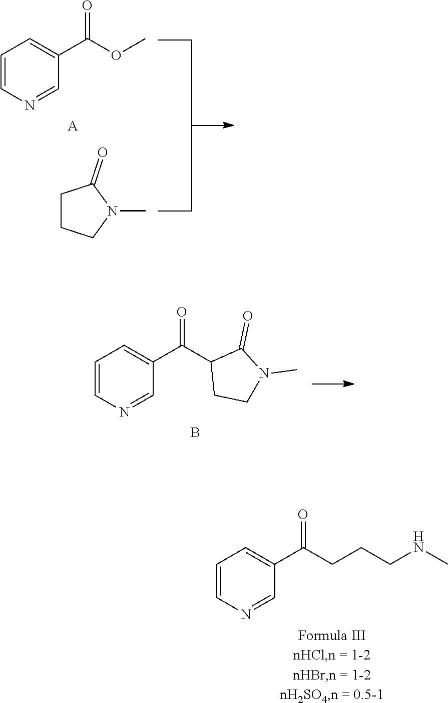

A method for synthesizing the compound A, and the method comprises: mixing nicotinic acid with methanol to form a mixture; and esterifying the mixture in a high acidic environment, thereby yielding the compound A.

In a class of this embodiment, a method for synthesizing (S)-nicotine comprises:
mixing nicotinic acid with methanol to form a mixture; and esterifying the mixture in a high acidic environment, thereby obtaining a compound A; mixing the compound A with N-methylpyrrolidone and the organic base to yield the compound D, or mixing the compound A with N-methylpyrrolidone and the organic base, and further adding a first acid for neutralization, to yield the compound B; mixing the compound B or the compound D with a second acid, thereby producing the salt of the compound of Formula III; desalting and cyclizing the salt of the compound of Formula III, and reducing a resulting product using a biocatalytic method or a chiral metal catalyst to obtain (S)-nicotine; the reaction equations are as follows:

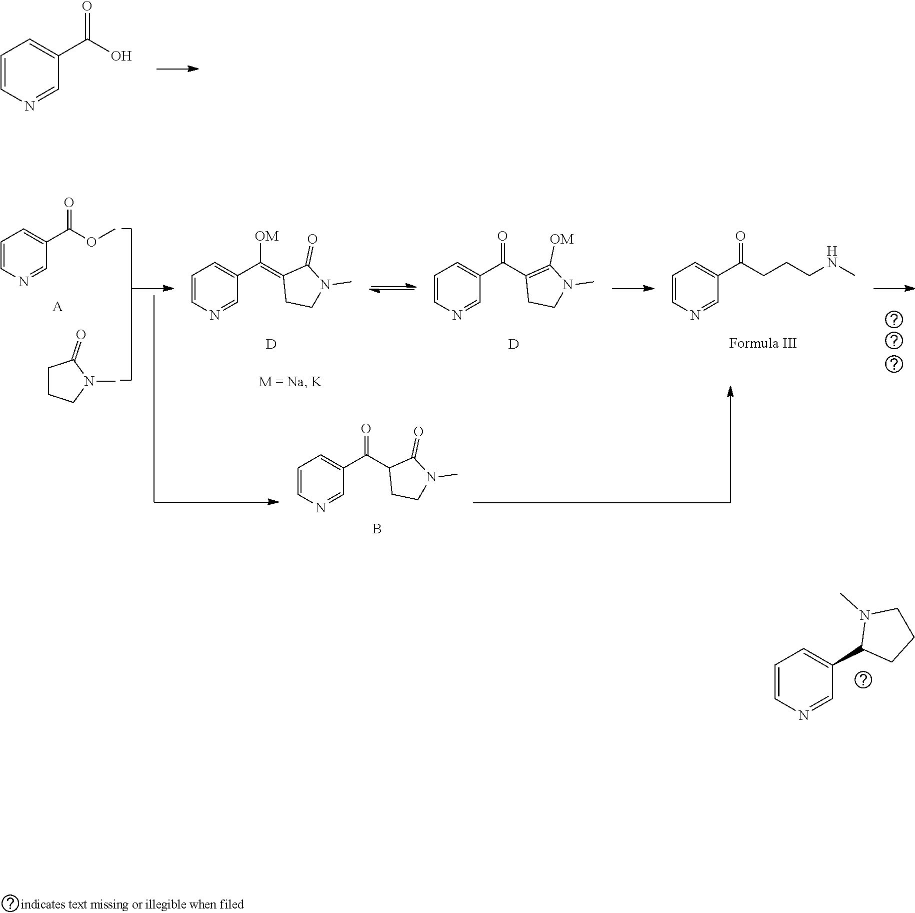

The following advantages are associated with the disclosure:

Considering that the majority of previously disclosed methods for preparing (S)-nicotine typically involve a methylation step to yield (S)-nicotine, or when enzymatic catalysis is used, methylation is still a requirement, indicating that the available methods for preparing (S)-nicotine have limitations. The disclosure provides a method for the synthesis of (S)-nicotine that removes the need for methylation. Specifically, the method is achieved by reducing the alkene compound as shown in Formula I and/or the iminium cation compound as shown in Formula II. The method is simple, safe, reliable, and yields both high purity and high quantities of (S)-nicotine production, thereby enhancing the synthetic options available for (S)-nicotine production.

### BRIEF DESCRIPTION OF THE DRAWINGS

FIG. 1 is a nuclear magnetic resonance (NMR) spectrum of (S)-nicotine prepared according to one example of the disclosure;

FIG. 2 is a NMR spectrum of a hydrochloride salt of a compound C prepared according to one example of the disclosure; and

FIG. 3 is a NMR spectrum of a compound B prepared according to one example of the disclosure.

### DETAILED DESCRIPTION

To further illustrate the disclosure, embodiments detailing a method for preparing (S)-nicotine are described below. It should be noted that the following embodiments are intended to describe and not to limit the disclosure.

In the following embodiments, the purity of a Compound A is determined using high-performance liquid chromatography (HPLC), and a method for calculating the yield of the Compound A is as follows:

$$Yield = \frac{Product weight \times Molecular weight of reactant}{Reactant weight \times Molecular weight of product} \times 100 \%$$

the purity of a Compound B is determined using high-performance liquid chromatography (HPLC), and a method for calculating the yield of the Compound B is as follows:

$$Yield = \frac{Product weight \times Molecular weight of reactant}{Reactant weight \times Molecular weight of product} \times 100 \%$$

the purity of a Compound C or a salt thereof is determined using high-performance liquid chromatography (HPLC), and a method for calculating the yield of the Compound C or a salt thereof is as follows:

$$Yield = \frac{Product weight \times Molecular weight of reactant}{Reactant weight \times Molecular weight of product} \times 100 \%$$

the purity and the optical purity of (S)-nicotine are determined using high-performance liquid chromatography (HPLC), and a method for calculating the yield of (S)-nicotine is as follows:

$$Yield = \frac{Product weight \times Molecular weight of reactant}{Reactant weight \times Molecular weight of product} \times 100 \%$$

### Preparation Example 1-1

### Preparation of Compound A

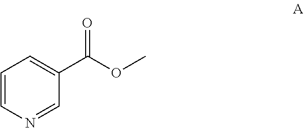

50 g of nicotinic acid and 250 g of methanol were added into a 500 mL reaction vessel. With continuous stirring, 60 g of concentrated sulfuric acid was gradually added to the reaction vessel to form a mixture. The temperature was then raised, and the mixture underwent reflux for 18 hours and was cooled to 45° C. Subsequently, the pressure was reduced to concentrate the mixture, removing methanol. After distilling off any residue, the concentrated residue was cooled to 20° C. Then, 250 g of ethyl acetate and 250 g of water was added and stirred until complete dissolution. The pH of the resulting mixture was adjusted to 7.5 using a 20% sodium hydroxide solution, and the resulting mixture was allowed to separate into an organic layer and an aqueous layer. The organic layer was collected. The aqueous layer was extracted once with 250 g of ethyl acetate, combined with the organic layer, and dried using anhydrous sodium sulfate. The solvent was removed under reduced pressure, thereby yielding 55.2 g of a colorless, transparent liquid with a purity of 99% and a yield of 99%. The colorless and transparent liquid solidified upon cooling.

### Preparation Example 1-2

### Preparation of Compound A

50 g of nicotinic acid and 250 g of methanol were added into a 500 mL reaction vessel. With continuous stirring, 120.5 g of thionyl chloride (2.5 equivalents) was added dropwise to the reaction vessel to form a mixture. The temperature was then raised, and the mixture underwent reflux for 9 hours and was cooled to 40° C. Subsequently, the pressure was reduced to concentrate the mixture, removing methanol. After distilling off any residue, the concentrated residue was cooled to 10° C. Then, 200 g of dichloromethane and 100 g of water was added and stirred until fully dissolved. The pH of the resulting mixture was adjusted to 8 using a 15% sodium hydroxide solution, and the resulting mixture was allowed to separate into an organic layer and an aqueous layer. The organic layer was collected. The aqueous layer was extracted once with 200 g of dichloromethane, combined with the organic layer, washed with a 5% sodium hydroxide solution, and dried using anhydrous sodium sulfate. The solvent was removed under reduced pressure, thereby yielding 44.8 g of a light yellow, transparent liquid with a purity of 99% and a yield of 81%. The liquid solidified upon cooling.

### Preparation Example 1-3

### Preparation of Compound A

5 g of nicotinic acid, 5.2 g of trimethyl orthoformate (1.2 equivalents), 25 g of methanol, and 0.48 g of zirconium tetrachloride (0.05 equivalents) were added into a 100 mL reaction vessel. With continuous stirring, the temperature was then raised, and the mixture underwent reflux for 17 hours and was cooled to 25° C. Then, 0.55 g of sodium ethoxide (0.2 equivalents) was added to neutralize the cooled mixture, followed by filtration. The filtrate was subjected to vacuum concentration, removing methanol, thereby yielding 5.5 g of a colorless, transparent liquid with a purity of 98.1% and a yield of 99%. The liquid solidified upon cooling.

### Preparation Example 1-4

### Preparation of Compound A

5 g of nicotinic acid, 5.2 g of trimethyl orthoformate (1.2 equivalents), 25 g of methanol, and 0.1 g of zirconium tetrachloride (0.01 equivalents) were added into a 100 mL reaction vessel. With continuous stirring, the temperature was then raised, and the mixture underwent reflux for 36 hours and cooled to 30° C. Then, 0.11 g of sodium ethoxide (0.04 equivalents) was added to neutralize the cooled mixture, followed by filtration. The filtrate was subjected to vacuum concentration, removing methanol, thereby yielding 5.3 g of a colorless, transparent liquid with a purity of 91.6% and a yield of 95%. The liquid solidified upon cooling.

### Preparation Example 1-5

### Preparation of Compound A

5 g of nicotinic acid, 5.2 g of trimethyl orthoformate (1.2 equivalents), 25 g of methanol, and 0.19 g of zirconium tetrachloride (0.02 equivalents) were added into a 100 mL reaction vessel. With continuous stirring, the temperature was then raised, and the mixture underwent reflux for 30 hours and cooled to 20° C. Then, 0.22 g of sodium ethoxide (0.08 equivalents) was added to neutralize the cooled mixture, followed by filtration. The filtrate was subjected to vacuum concentration, removing methanol, thereby yielding 5.4 g of a colorless, transparent liquid with a purity of 93.9% and a yield of 97%. The liquid solidified upon cooling.

### Preparation Example 1-6

### Preparation of Compound A

200 g of nicotinic acid, 207 g of trimethyl orthoformate (1.2 equivalents), 1000 g of methanol, and 9.5 g of zirconium tetrachloride (0.025 equivalents) were added into a 2 L reaction vessel. With continuous stirring, the temperature was raised, and the mixture underwent reflux for 20 hours and cooled to 25° C. Then, 11.06 g of sodium ethoxide (0.1 equivalents) was added to neutralize the cooled mixture, followed by filtration. The filtrate was subjected to vacuum concentration, removing methanol, thereby yielding 220.6 g of a colorless, transparent liquid with a purity of 98.5% and a yield of 99%. The liquid solidified upon cooling.

### Preparation Example 2-1

### Preparation of Compound B

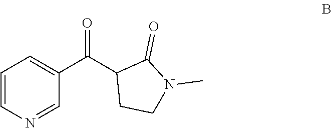

102.0 g of the Compound A prepared in Preparation Example 1-1, 88.5 g of N-methylpyrrolidone (1.2 equivalents), 1.2 kg of toluene, and 133.6 g of potassium tert-butoxide (1.6 equivalents) were added into a reaction vessel. With continuous stirring, the temperature was raised, and the mixture underwent reflux. Once the reaction was completed, the mixture was cooled to 25° C., neutralized to a pH of 7.5 using 5% hydrochloric acid, followed by separation into an organic layer and an aqueous layer. The aqueous layer was extracted with dichloromethane, combined with the organic layer, and subjected to vacuum concentration to remove the solvent, thereby yielding 121.0 g of a yellow-brown liquid, identified as a Compound B, with a purity of 94% and a yield of 80%. No further purification steps were necessary, and the Compound B was directly advanced to the subsequent stage of the reaction.

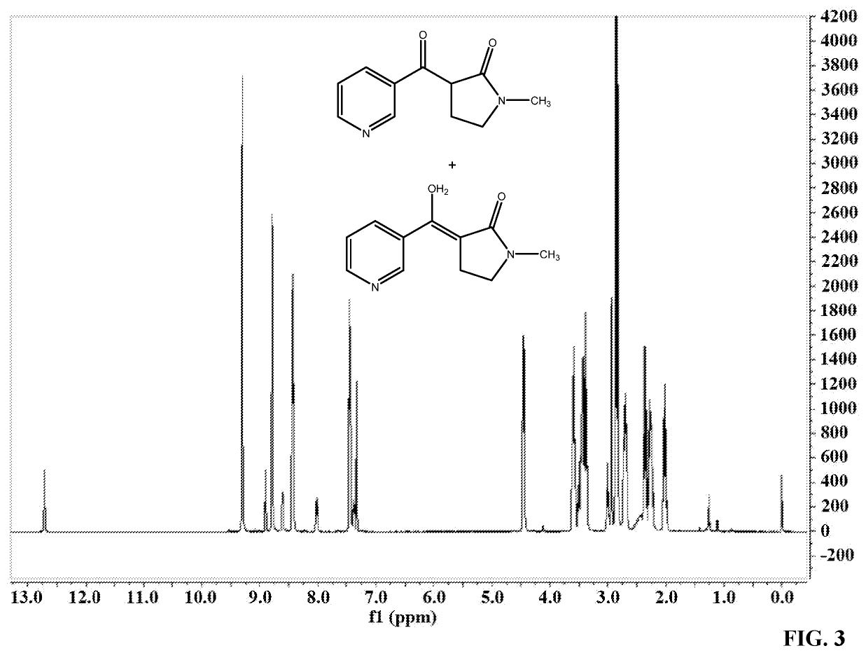

The resulting Compound B, in a crude state, was purified by column chromatography and analyzed using nuclear magnetic resonance (NMR). The NMR analysis revealed the coexistence of both keto and enol forms, with the keto form being predominant at a ratio of 10:1. The NMR hydrogen spectrum data for the keto form of the Compound B were presented as follows: δ ppm (400 MHz, CDCl₃) 9.30 (s, 1H), 8.77-8.80 (m, 1H), 8.44 (1H, dt, J=8.0, 2.0 Hz), 7.43-7.47 (m, 1H), 4.46 (1H, dd, J=8.8, 3.2 Hz), 3.36-3.46 (m, 2H), 2.87 (s, 3H), 2.67-2.75 (m, 1H), 2.22-2.32 (m, 1H). The NMR hydrogen spectrum was depicted in FIG. 3.

### Preparation Example 2-2

### Preparation of Compound B

102.0 g of the Compound A prepared in Preparation Example 1-2, 77.4 g of N-methylpyrrolidone (1.05 equivalents), 1.2 kg of toluene, and 91.8 g of potassium tert-butoxide (1.1 equivalents) were added into a reaction vessel. With continuous stirring, the temperature was raised, and the mixture underwent reflux. Once the reaction was completed, the mixture was cooled to 20° C., neutralized to a pH of 7 using 5% hydrochloric acid, followed by separation into an organic layer and an aqueous layer. The aqueous layer was extracted with toluene, combined with the organic layer, and subjected to vacuum concentration to remove the solvent, thereby yielding 118.0 g of a yellow-brown liquid, identified as Compound B, with a purity of 93.5% and a yield of 78%. No further purification steps were necessary, and the Compound B was directly advanced to the subsequent stage of the reaction.

The resulting Compound B, in a crude state, was purified by column chromatography and analyzed using nuclear magnetic resonance (NMR). The NMR analysis revealed the coexistence of both keto and enol forms, with the keto form being predominant at a ratio of 10:1. The NMR hydrogen spectrum data for the keto form of the Compound B were presented as follows: δ ppm (400 MHz, CDCl₃) 9.30 (s, 1H), 8.77-8.80 (m, 1H), 8.44 (1H, dt, J=8.0, 2.0 Hz), 7.43-7.47 (m, 1H), 4.46 (1H, dd, J=8.8, 3.2 Hz), 3.36-3.46 (m, 2H), 2.87 (s, 3H), 2.67-2.75 (m, 1H), 2.22-2.32 (m, 1H).

### Preparation Example 2-3

### Preparation of Compound B

102.0 g of the Compound A prepared in Preparation Example 1-3, 88.5 g of N-methylpyrrolidone (1.2 equivalents), 1.2 kg of toluene, and 114.4 g of sodium tert-butoxide (1.6 equivalents) were added into a reaction vessel. With continuous stirring, the temperature was raised, and the mixture underwent reflux. Once the reaction was completed, the mixture was cooled to 30° C., and neutralized to a pH of 8 using 5% hydrochloric acid, followed by separation into an organic layer and an aqueous layer. The aqueous layer was extracted with ethyl acetate, combined with the organic layer, and subjected to vacuum concentration to remove the solvent, thereby yielding 117.8 g of a yellow-brown liquid, identified as a Compound B, with a purity of 95% and a yield of 78%. No further purification steps were necessary, and the Compound B was directly advanced to the subsequent stage of the reaction.

The resulting Compound B, in a crude state, was purified by column chromatography and analyzed using nuclear magnetic resonance (NMR). The NMR analysis revealed the coexistence of both keto and enol forms, with the keto form being predominant at a ratio of 10:1. The NMR hydrogen spectrum data for the keto form of the Compound B were presented as follows: δ ppm (400 MHz, CDCl₃) 9.30 (s, 1H), 8.77-8.80 (m, 1H), 8.44 (1H, dt, J=8.0, 2.0 Hz), 7.43-7.47 (m, 1H), 4.46 (1H, dd, J=8.8, 3.2 Hz), 3.36-3.46 (m, 2H), 2.87 (s, 3H), 2.67-2.75 (m, 1H), 2.22-2.32 (m, 1H).

### Preparation Example 2-4

### Preparation of Compound B

102.0 g of the Compound A prepared in Preparation Example 1-4, 77.4 g of N-methylpyrrolidone (1.05 equivalents), 1.2 kg of toluene, and 78.6 g of sodium tert-butoxide (1.1 equivalents) were added into a reaction vessel. With continuous stirring, the temperature was raised, and the mixture underwent reflux. Once the reaction was completed, the mixture was cooled to 25° C., and neutralized to a pH of 7.5 using 5% hydrochloric acid, followed by separation into an organic layer and an aqueous layer. The aqueous layer was extracted with toluene, combined with the organic layer, and subjected to vacuum concentration to remove the solvent, thereby yielding 108.7 g of a yellow-brown liquid, identified as a Compound B, with a purity of 93% and a yield of 72%. No further purification steps were necessary, and the Compound B was directly advanced to the subsequent stage of the reaction.

The resulting Compound B, in a crude state, was purified by column chromatography and analyzed using nuclear magnetic resonance (NMR). The NMR analysis revealed the coexistence of both keto and enol forms, with the keto form being predominant at a ratio of 10:1. The NMR hydrogen spectrum data for the keto form of the Compound B were presented as follows: δ ppm (400 MHz, CDCl₃) 9.30 (s, 1H), 8.77-8.80 (m, 1H), 8.44 (1H, dt, J=8.0, 2.0 Hz), 7.43-7.47 (m, 1H), 4.46 (1H, dd, J=8.8, 3.2 Hz), 3.36-3.46 (m, 2H), 2.87 (s, 3H), 2.67-2.75 (m, 1H), 2.22-2.32 (m, 1H).

### Preparation Example 2-5

### Preparation of Compound B

102.0 g of the Compound A prepared in Preparation Example 1-5, 88.5 g of N-methylpyrrolidone (1.2 equivalents), 1.2 kg of toluene, and 81.0 g of sodium ethoxide (1.6 equivalents) were added into a reaction vessel. With continuous stirring, the temperature was raised, and the mixture underwent reflux for 16 hours. Once the reaction was completed, the mixture was cooled to 25° C., and neutralized to a pH of 7.5 using 5% hydrochloric acid, followed by separation into an organic layer and an aqueous layer. The aqueous layer was extracted with toluene, combined with the organic layer, and subjected to vacuum concentration to remove the solvent, thereby yielding 127.0 g of a yellow-brown liquid, identified as a Compound B, with a purity of 92% and a yield of 84%. No further purification steps were necessary, and the Compound B was directly advanced to the subsequent stage of the reaction.

The resulting Compound B, in a crude state, was purified by column chromatography and analyzed using nuclear magnetic resonance (NMR). The NMR analysis revealed the coexistence of both keto and enol forms, with the keto form being predominant at a ratio of 10:1. The NMR hydrogen spectrum data for the keto form of the Compound B were presented as follows: δ ppm (400 MHz, CDCl₃) 9.30 (s, 1H), 8.77-8.80 (m, 1H), 8.44 (1H, dt, J=8.0, 2.0 Hz), 7.43-7.47 (m, 1H), 4.46 (1H, dd, J=8.8, 3.2 Hz), 3.36-3.46 (m, 2H), 2.87 (s, 3H), 2.67-2.75 (m, 1H), 2.22-2.32 (m, 1H).

### Preparation Example 2-6

### Preparation of Compound B

102.0 g of the Compound A prepared in Preparation Example 1-6, 88.5 g of N-methylpyrrolidone (1.2 equivalents), 1.2 kg of toluene, and 47.6 g of sodium hydride (1.6 equivalents, 60% sodium hydride in oil) were added into a reaction vessel. With continuous stirring, the temperature was raised, and the mixture underwent reflux. Once the reaction was completed, the mixture was cooled to 25° C., and neutralized to a pH of 7 using 5% hydrochloric acid, followed by separation into an organic layer and an aqueous layer. The aqueous layer was extracted with methyl tert-butyl ether, combined with the organic layer, and subjected to vacuum concentration to remove the solvent, thereby yielding 111.0 g of a yellow-brown liquid, identified as a Compound B, with a purity of 90% and a yield of 70%. No further purification steps were necessary, and the Compound B was directly advanced to the subsequent stage of the reaction.

The resulting Compound B, in a crude state, was purified by column chromatography and analyzed using nuclear magnetic resonance (NMR). The NMR analysis revealed the coexistence of both keto and enol forms, with the keto form being predominant at a ratio of 10:1. The NMR hydrogen spectrum data for the keto form of the Compound B were presented as follows: δ ppm (400 MHz, CDCl₃) 9.30 (s, 1H), 8.77-8.80 (m, 1H), 8.44 (1H, dt, J=8.0, 2.0 Hz), 7.43-7.47 (m, 1H), 4.46 (1H, dd, J=8.8, 3.2 Hz), 3.36-3.46 (m, 2H), 2.87 (s, 3H), 2.67-2.75 (m, 1H), 2.22-2.32 (m, 1H).

### Preparation Example 3-1

### Preparation of a Hydrochloride Salt of Compound C Using Compound B

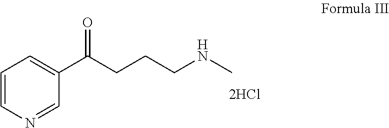

100.0 g of the Compound B prepared in Preparation Example 2-1 and 500.0 g of concentrated hydrochloric acid were added into a reaction vessel. With continuous stirring, the temperature was raised, and the mixture underwent reflux. Once the reaction was completed, the mixture was cooled to 55° C. and subjected to vacuum concentration to remove the solvent. 300.0 g of methanol was added to the resulting residue, cooled to 4° C., and subjected to filtration, thereby yielding 92.2 g of a nearly white solid, identified as a Compound C, with a purity of 99% and a yield of 75%.

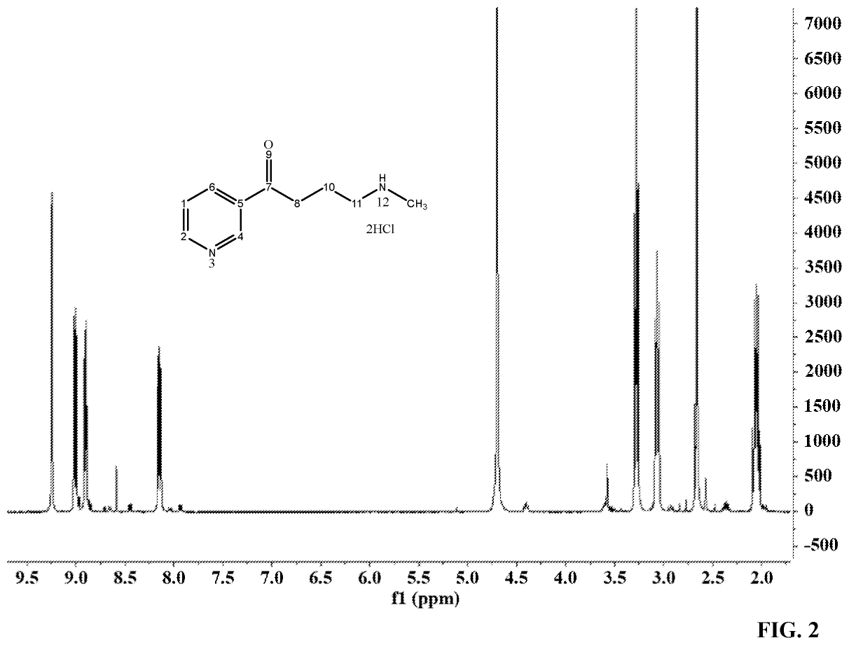

Upon analysis, the proton nuclear magnetic resonance (¹H-NMR) spectrum of the Compound C as shown in Formula III matched the provided structure, and the spectral data were as follows: ¹H-NMR (400 MHz, D₂O): δ ppm 9.25 (m, 1H), 9.0 (dt, J=8.4 Hz, 1.6 Hz, 1H), 8.90-8.91 (m, 1H), 8.13-8.17 (m, 1H), 3.28 (t, J=6.8 Hz, 2H), 3.06 (t, J=8.0 Hz, 2H), 2.66 (s, 3H), 2.02-2.09 (m, 2H). The spectrum was depicted in FIG. 2.

### Preparation Example 3-2

### Preparation of Compound C Using Compound B

100.0 g of the Compound B prepared in Preparation Example 2-2 and 100.0 g of concentrated hydrochloric acid were added into a reaction vessel. With continuous stirring, the temperature was raised, and the mixture underwent reflux. Once the reaction was completed, the mixture was cooled to 50° C. and subjected to vacuum concentration to remove the solvent. 100.0 g of methanol was added to the resulting residue, cooled to 8° C., and subjected to filtration, thereby yielding 95.9 g of a nearly white solid, identified as a Compound C, with a purity of 98.5% and a yield of 78%.

Upon analysis, the proton nuclear magnetic resonance (¹H-NMR) spectrum of the Compound C as shown in Formula III matched the provided structure, and the spectral data were as follows: ¹H-NMR (400 MHz, D₂O): δ ppm 9.25 (m, 1H), 9.0 (dt, J=8.4 Hz, 1.6 Hz, 1H), 8.90-8.91 (m, 1H), 8.13-8.17 (m, 1H), 3.28 (t, J=6.8 Hz, 2H), 3.06 (t, J=8.0 Hz, 2H), 2.66 (s, 3H), 2.02-2.09 (m, 2H).

### Preparation Example 3-3

### Preparation of Compound C Using Compound B

100.0 g of the Compound B prepared in Preparation Example 2-3 and 300.0 g of concentrated hydrochloric acid were added into a reaction vessel. With continuous stirring, the temperature was raised, and the mixture underwent reflux. Once the reaction was completed, the mixture was cooled to 50° C. and subjected to vacuum concentration to remove the solvent. 300.0 g of ethanol was added to the resulting residue, cooled to 0° C., and subjected to filtration, thereby yielding 100.8 g of a nearly white solid, identified as a Compound C, with a purity of 99% and a yield of 82%.

Upon analysis, the proton nuclear magnetic resonance (¹H-NMR) spectrum of the Compound C as shown in Formula III matched the provided structure, and the spectral data were as follows: ¹H-NMR (400 MHz, D₂O): δ ppm 9.25 (m, 1H), 9.0 (dt, J=8.4 Hz, 1.6 Hz, 1H), 8.90-8.91 (m, 1H), 8.13-8.17 (m, 1H), 3.28 (t, J=6.8 Hz, 2H), 3.06 (t, J=8.0 Hz, 2H), 2.66 (s, 3H), 2.02-2.09 (m, 2H).

### Preparation Example 3-4

### Preparation of Compound C Using Compound B

100.0 g of the Compound B prepared in Preparation Example 2-4 and 300.0 g of concentrated hydrochloric acid were added into a reaction vessel. With continuous stirring, the temperature was raised, and the mixture underwent reflux. Once the reaction was completed, the mixture was cooled to 55° C. and subjected to vacuum concentration to remove the solvent. 500.0 g of isopropanol was added to the resulting residue, cooled to 4° C., and subjected to filtration, thereby yielding 93.4 g of a nearly white solid, identified as a Compound C, with a purity of 97.5% and a yield of 76%.

Upon analysis, the proton nuclear magnetic resonance (¹H-NMR) spectrum of the Compound C as shown in Formula III matched the provided structure, and the spectral data were as follows: ¹H-NMR (400 MHz, D₂O): δ ppm 9.25 (m, 1H), 9.0 (dt, J=8.4 Hz, 1.6 Hz, 1H), 8.90-8.91 (m, 1H), 8.13-8.17 (m, 1H), 3.28 (t, J=6.8 Hz, 2H), 3.06 (t, J=8.0 Hz, 2H), 2.66 (s, 3H), 2.02-2.09 (m, 2H).

### Preparation Example 3-5

### Preparation of Compound C Using Compound B

100.0 g of the Compound B prepared in Preparation Example 2-5 and 500.0 g of concentrated hydrochloric acid were added into a reaction vessel. With continuous stirring, the temperature was raised, and the mixture underwent reflux. Once the reaction was completed, the mixture was cooled to 55° C. and subjected to vacuum concentration to remove the solvent. 100.0 g of isopropanol was added to the resulting residue, cooled to 4° C., and subjected to filtration, thereby yielding 103.3 g of a nearly white solid, identified as a Compound C, with a purity of 96% and a yield of 84%.

Upon analysis, the proton nuclear magnetic resonance (¹H-NMR) spectrum of the Compound C as shown in Formula III matched the provided structure, and the spectral data were as follows: ¹H-NMR (400 MHz, D₂O): δ ppm 9.25 (m, 1H), 9.0 (dt, J=8.4 Hz, 1.6 Hz, 1H), 8.90-8.91 (m, 1H), 8.13-8.17 (m, 1H), 3.28 (t, J=6.8 Hz, 2H), 3.06 (t, J=8.0 Hz, 2H), 2.66 (s, 3H), 2.02-2.09 (m, 2H).

### Preparation Example 4-1

Compound D was first prepared from Compound A as a starting material, followed by preparation of Compound C.

120.0 g of the Compound A prepared in Preparation Example 1-1, 96.2 g of N-methylpyrrolidone, 1.6 kg of toluene, and 147 g of potassium tert-butoxide were added into a reaction vessel. With continuous stirring, the temperature was raised, and the mixture underwent reflux. Once the reaction was completed, the mixture was cooled to 25° C., and subjected to filtration, thereby yielding a Compound D (potassium salt). The Compound D was directly advanced to the subsequent stage of the reaction.

The Compound D (potassium salt) was added to 650 g of concentrated hydrochloric acid, and the temperature was raised to 100° C. Once the reaction was completed, vacuum distillation was employed to remove most of the water from the resulting mixture. Then, 650 mL of 95% ethanol was added to the residue, followed by thorough stirring. The temperature was reduced to 10° C., and the mixture was subjected to filtration and drying, thereby yielding 86 g of a nearly white solid, identified as a Compound C, with a yield of 55.2% and a purity of 97.2%.

Upon analysis, the nuclear magnetic resonance (¹H-NMR) spectrum of the compound C as shown in Formula III matched the provided structure, and the spectral data were as follows: ¹H-NMR (400 MHz, D₂O): δ ppm 9.25 (m, 1H), 9.0 (dt, J=8.4 Hz, 1.6 Hz, 1H), 8.90-8.91 (m, 1H), 8.13-8.17 (m, 1H), 3.28 (t, J=6.8 Hz, 2H), 3.06 (t, J=8.0 Hz, 2H), 2.66 (s, 3H), 2.02-2.09 (m, 2H).

### Preparation Example 4-2

Compound D was first prepared from Compound A as a starting material, followed by preparation of Compound C.

153.0 g of the Compound A prepared in Preparation Example 1-2, 165.0 g of N-methylpyrrolidone, 2 kg of toluene, and 214.5 g of potassium tert-butoxide were added into a reaction vessel. With continuous stirring, the temperature was raised, and the mixture underwent reflux. Once the reaction was completed, the mixture was cooled to 15° C., and subjected to filtration, thereby yielding a Compound D (potassium salt). The Compound D was directly advanced to the subsequent stage of the reaction.

The Compound D (potassium salt) was added to 960 g of concentrated hydrochloric acid, and the temperature was raised to 100° C. Once the reaction was completed, vacuum distillation was employed to remove most of the water from the resulting mixture. Then, 800 mL of isopropanol was added to the residue, followed by thorough stirring. The temperature was reduced to 20° C., and the mixture was subjected to filtration and drying, thereby yielding 127 g of a nearly white solid, identified as the Compound C, with a yield of 63.5% and a purity of 96.7%.

Upon analysis, the nuclear magnetic resonance (¹H-NMR) spectrum of the compound C as shown in Formula III matched the provided structure, and the spectral data were as follows: ¹H-NMR (400 MHz, D₂O): δ ppm 9.25 (m, 1H), 9.0 (dt, J=8.4 Hz, 1.6 Hz, 1H), 8.90-8.91 (m, 1H), 8.13-8.17 (m, 1H), 3.28 (t, J=6.8 Hz, 2H), 3.06 (t, J=8.0 Hz, 2H), 2.66 (s, 3H), 2.02-2.09 (m, 2H).

### Preparation Example 5-1

### Preparation of Glucose Dehydrogenase Comprising an Amino Acid Sequence Shown in SEQ ID NO: 1

The amino acid sequence of glucose dehydrogenase, derived from Priestia megaterium (NCBI accession number AU012718.1) (SEQ ID NO: 1) was submitted to Nanjing Kingsray Corporation for codon optimization and full gene synthesis. Subsequently, the amino acid sequence was ligated into the plasmid pET30a(+) and transformed into Escherichia coli BL21 (DE3) competent cells, thereby obtaining recombinant bacteria containing a glucose dehydrogenase gene.

The recombinant bacteria were inoculated into 5 mL of LB liquid medium containing 50 μg/mL kanamycin and cultured overnight at 37° C. Then, 1 mL of the bacterial culture was transferred into 125 mL of LB liquid medium containing 50 μg/mL kanamycin and incubated at 37° C. for 3 hours; subsequently, 125 μL of 1M IPTG was added, and the culture was induced overnight at 25° C. The cells were harvested by centrifugation (at 4000 rpm and 4° C. for 10 min), resuspended in phosphate buffer (pH=7.0) at four times the original volume, subjected to sonication for cell disruption, and then centrifuged (at 4000 rpm and 4° C. for 10 min) to collect the supernatant. The supernatant was freeze-dried to obtain the powder of glucose dehydrogenase enzyme (denoted as Enzyme 2).

### Preparation Example 5-2

Preparation of eleven different imine reductases, denoted as Enzymes 1, 3-11, and 13.

The amino acid sequences of imine reductases, as reported on NCBI (with information provided in the table below as SEQ ID NO: 2-12), were submitted to Nanjing Kingsray Corporation for codon optimization and full gene synthesis. Subsequently, the amino acid sequences were ligated into plasmid pET30a(+) and transformed into Escherichia coli BL21 (DE3) competent cells, thereby obtaining recombinant bacteria containing an imine reductase gene.

| Amino acid | NCBI accession | |
|---|---|---|
| sequence | number | Source |
| SEQ ID NO: 2 | WP_201992507.1 | Aeromonas veronii |
| SEQ ID NO: 3 | WP_139729886.1 | Aeromonas sobria |
| SEQ ID NO: 4 | WP_010862236.1 | Plesiomonas shigelloides |
| SEQ ID NO: 5 | WP_064339362.1 | Aeromonas veronii |
| SEQ ID NO: 6 | QNF15299.1 | Aeromonas jandaei |
| SEQ ID NO: 7 | WP_073480922.1 | Streptoalloteichus hindustanus |
| SEQ ID NO: 8 | WP_015347361.1 | Myxococcus stipitatus |
| SEQ ID NO: 9 | WP_198539790.1 | Rhizobium sp. |
| SEQ ID NO: 10 | WP_073459042.1 | Pseudonocardia thermophila |
| SEQ ID NO: 11 | WP_020496004.1 | Sciscionella marina |
| SEQ ID NO: 12 | WP_005335883.1 | Aeromonas veronii |

The recombinant bacteria were inoculated into 5 mL of LB liquid medium containing 50μg/mL kanamycin and cultured overnight at 37° C. Subsequently, 1 mL of the bacterial culture was transferred into 125 mL of LB liquid medium containing 50 μg/mL kanamycin and incubated at 37° C. for 3 hours; then, 125 μL of 1M IPTG was added, and the culture was induced for 16 hours at 25° C. The cells were harvested by centrifugation (at 4000 rpm, 4° C., for 10 min), resuspended in phosphate buffer (pH=7.0) at a volume four times the original, subjected to sonication for cell disruption, and then centrifuged again (at 4000 rpm, 4° C., for 10 min) to collect the supernatant. The obtained supernatant was freeze-dried to yield the powder of the imine reductase.

Ten imine reductases, denoted as Enzymes 14-23, were also prepared by performing site-directed mutagenesis on the amino acid sequence as shown in SEQ ID NO: 12, thereby obtaining the imine reductases containing the amino acid sequences as shown in SEQ ID NOs: 13-22. The amino acid sequences were submitted to Nanjing Kingsray Corporation for codon optimization and full gene synthesis. Subsequently, the amino acid sequences were ligated into plasmid pET30a(+) and transferred into Escherichia coli BL21 (DE3) competent cells, thereby obtaining recombinant bacteria containing the imine reductase genes. The same operations were performed to ultimately obtain the powder of imine reductase.

### Preparation Example 5-3

### Preparation of Enzyme 12

The amino acid sequence (SEQ ID NO: 2) of imine reductase and the amino acid sequence (SEQ ID NO: 1) of glucose dehydrogenase were sequentially cloned into the plasmid pETDuet-1. The recombinant plasmid was then transferred into Escherichia coli BL21 (DE3) competent cells, thereby obtaining recombinant bacteria containing both the imine reductase gene and the glucose dehydrogenase gene.

The recombinant bacteria were inoculated into 5 mL of LB liquid medium containing 100 μg/mL ampicillin and cultured overnight at 37° C. Subsequently, 1 mL of the bacterial culture was transferred into 125 mL of LB liquid medium containing 100 μg/mL ampicillin and incubated at 37° C. for 3 hours; then, 125 μL of 1M IPTG was added, and the culture was induced overnight at 25° C. The cells were harvested by centrifugation (at 4000 rpm an 4° C. for 10 min), resuspended in phosphate buffer (pH=7.0) at a volume four times the original, subjected to sonication for cell disruption, and then centrifuged again (at 4000 rpm and 4° C. for 10 min) to collect the supernatant. The obtained supernatant was freeze-dried to yield the powder of Enzyme 12.

### Example 1

Preparation of (S)-nicotine Through a Reduction Process Using a Chiral Metal Catalyst

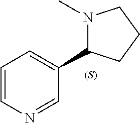

1.25 g of the Compound C prepared in Preparation Example 3-1, 50 mL of methanol, 1.0 g of triethylamine, 16.7 mg of chloro (1,5-cyclooctadiene)iridium (I) dimer as a chiral metal catalyst, and 30 mg of (S)-(−)-1-((R)-2-diphenylphosphino)ferrocenyl)ethyldi-t-butylphosphine as a ligand, were added to a 100 mL hydrogenation reactor. Hydrogen gas was then introduced into the hydrogenation reactor to maintain a pressure of 2.5 MPa, and the temperature was controlled at 60° C. When the reaction was completed, the solvent was removed through concentration, and the residue was slurried in n-heptane, filtered, and concentrated to obtain (S)-nicotine, with a purity of 96% and an optical purity of 72%.

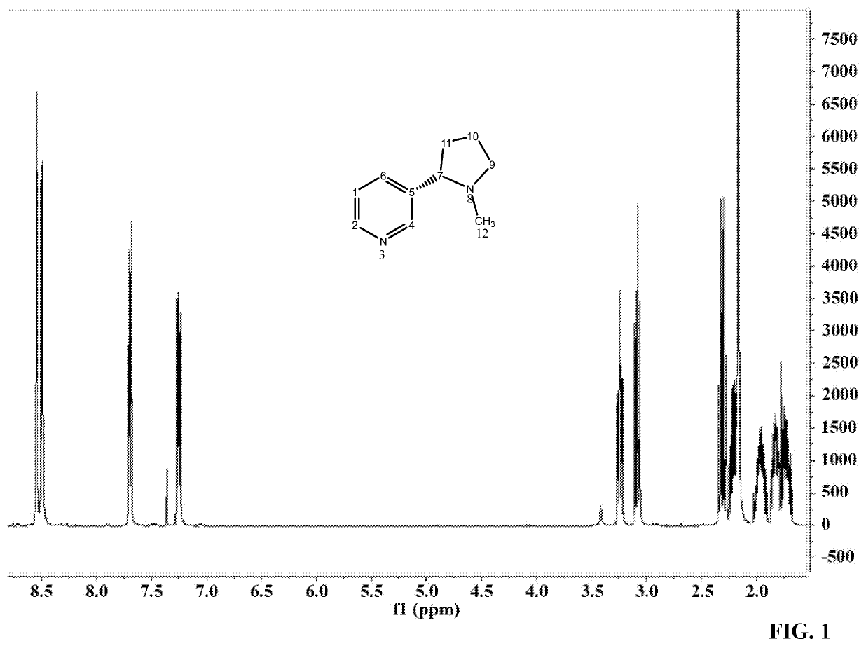

The (S)-nicotine obtained was purified by column chromatography and characterized using nuclear magnetic resonance (NMR), as shown in FIG. 1. The NMR data are as follows: ¹H-NMR (400 MHz, CDCl₃): δ ppm 8.54 (1H, d), 8.50 (1H, dd), 7.70 (1H, dt), 7.24-7.27 (1H, m), 3.22-3.27 (1H, m), 3.08 (1H, t), 2.27-2.34 (1H, m), 2.17-2.24 (1H, m), 2.16 (3H, m), 1.91-2.02 (1H, m), 1.79-1.87 (1H, m), 1.68-1.76 (1H, m). The results confirm a successful synthesis of (S)-nicotine.

### Example 2

Preparation of (S)-nicotine Through a Reduction Process Using a Chiral Metal Catalyst

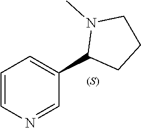

1.25 g of the Compound C prepared in Preparation Example 3-2, 50 mL of methanol, 1.0 g of triethylamine, 16.7 mg of chloro (1,5-cyclooctadiene)iridium (I) dimer as a chiral metal catalyst, and 27 mg of (+)-1,2-Bis((2S,5S)-2,5-diphenylphospholano)ethane as a ligand, were added to a 100 mL hydrogenation reactor. Hydrogen gas was then introduced into the hydrogenation reactor to maintain a pressure of 3.0 MPa, and the temperature was controlled at 60° C. When the reaction was completed, the solvent was removed through concentration, and the residue was slurried in n-heptane, filtered, and concentrated to obtain (S)-nicotine, with a purity of 94% and an optical purity of 80%.

The (S)-nicotine obtained was purified by column chromatography and was the characterized using nuclear magnetic resonance (NMR). The NMR data are as follows: ¹H-NMR (400 MHz, CDCl₃): δ ppm 8.54 (1H, d), 8.50 (1H, dd), 7.70 (1H, dt), 7.24-7.27 (1H, m), 3.22-3.27 (1H, m), 3.08 (1H, t), 2.27-2.34 (1H, m), 2.17-2.24 (1H, m), 2.16 (3H, m), 1.91-2.02 (1H, m), 1.79-1.87 (1H, m), 1.68-1.76 (1H, m). The results confirm a successful synthesis of (S)-nicotine.

### Example 3

Preparation of (S)-nicotine Through a Reduction Process Using a Chiral Metal Catalyst

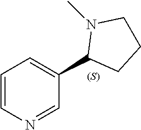

1.25 g of the Compound C prepared in Preparation Example 3-3, 50 mL of methanol, 1.0 g of triethylamine, 16.7 mg of chloro (1,5-cyclooctadiene)iridium (I) dimer as a chiral metal catalyst, and 38 mg of (R,R)-f-SpiroPhos as a ligand, were added to a 100 mL hydrogenation reactor. Hydrogen gas was then introduced into the hydrogenation reactor to maintain a pressure of 5.5 MPa, and the temperature was controlled at 80° C. When the reaction was completed, the solvent was removed through concentration, and the residue was slurried in n-heptane, filtered, and concentrated to obtain (S)-nicotine, with a purity of 90% and an optical purity of 88%.

The (S)-nicotine obtained was purified by column chromatography and the characterized using nuclear magnetic resonance (NMR). The NMR data are as follows: ¹H-NMR (400 MHz, CDCl₃): δ ppm 8.54 (1H, d), 8.50 (1H, dd), 7.70 (1H, dt), 7.24-7.27 (1H, m), 3.22-3.27 (1H, m), 3.08 (1H, t), 2.27-2.34 (1H, m), 2.17-2.24 (1H, m), 2.16 (3H, m), 1.91-2.02 (1H, m), 1.79-1.87 (1H, m), 1.68-1.76 (1H, m). The results confirm a successful synthesis of (S)-nicotine.

### Example 4

Preparation of (S)-nicotine Through a Reduction Process Using a Chiral Metal Catalyst

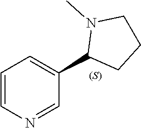

Example 4 is similar to Example 3, except for the following differences: 16.7 mg of chloro (1,5-cyclooctadiene)iridium (I) dimer and 38 mg of R,R)-f-SpiroPhos was replaced with 45 mg of Rh((R,R)-DIPAMP)(COD)BF₄, while keeping all other conditions unchanged. The resulting (S)-nicotine exhibited a purity of 45% and an optical purity of 27%.

The (S)-nicotine obtained was purified by column chromatography and characterized using nuclear magnetic resonance (NMR). The NMR data are as follows: ¹H-NMR (400 MHz, CDCl₃): δ ppm 8.54 (1H, d), 8.50 (1H, dd), 7.70 (1H, dt), 7.24-7.27 (1H, m), 3.22-3.27 (1H, m), 3.08 (1H, t), 2.27-2.34 (1H, m), 2.17-2.24 (1H, m), 2.16 (3H, m), 1.91-2.02 (1H, m), 1.79-1.87 (1H, m), 1.68-1.76 (1H, m). The results confirm a successful synthesis of (S)-nicotine.

### Example 5

Preparation of (S)-nicotine Through a Reduction Process Using a Chiral Metal Catalyst

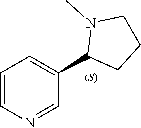

1.25 g of the Compound C prepared in Preparation Example 3-4, 60 mL of ethanol, 1.0 g of triethylamine, and 45 mg of Rh((R,R)-DIPAMP)(COD)BF₄ as a chiral metal catalyst, were added to a 100 mL hydrogenation reactor. Hydrogen gas was then introduced into the hydrogenation reactor to maintain a pressure of 4.0 MPa, and the temperature was controlled at 90° C. When the reaction was completed, the solvent was removed through concentration, and the residue was slurried in n-heptane, filtered, and concentrated to obtain (S)-nicotine, with a purity of 85% and an optical purity of 58%.

The (S)-nicotine obtained was purified by column chromatography and characterized using nuclear magnetic resonance (NMR). The NMR data are as follows: ¹H-NMR (400 MHz, CDCl₃): δ ppm 8.54 (1H, d), 8.50 (1H, dd), 7.70 (1H, dt), 7.24-7.27 (1H, m), 3.22-3.27 (1H, m), 3.08 (1H, t), 2.27-2.34 (1H, m), 2.17-2.24 (1H, m), 2.16 (3H, m), 1.91-2.02 (1H, m), 1.79-1.87 (1H, m), 1.68-1.76 (1H, m). The results confirm a successful synthesis of (S)-nicotine.

### Example 6

Preparation of (S)-nicotine Through a Reduction Process Using a Chiral Metal Catalyst

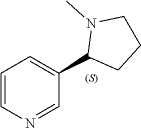

1.25 g of the Compound C prepared in Preparation Example 3-5, 50 mL of methanol, 1.0 g of N,N-diisopropylethylamine, and 39 mg of (−)-1,2-Bis((2S,5S)-2,5-dimethylphosphino)ethane(cyclooctadiene)rhodium(I) tetrafluoroborate as a chiral metal catalyst, were added to a 100 mL hydrogenation reactor. Hydrogen gas was then introduced into the hydrogenation reactor to maintain a pressure of 1.5 MPa, and the temperature was controlled at 100° C. When the reaction was completed, the solvent was removed through concentration, and the residue was slurried in methyl tert-butyl ether, filtered, and concentrated to obtain (S)-nicotine, with a purity of 64% and an optical purity of 82%.

The (S)-nicotine obtained was purified by column chromatography and characterized using nuclear magnetic resonance (NMR). The NMR data are as follows: ¹H-NMR (400 MHz, CDCl₃): δ ppm 8.54 (1H, d), 8.50 (1H, dd), 7.70 (1H, dt), 7.24-7.27 (1H, m), 3.22-3.27 (1H, m), 3.08 (1H, t), 2.27-2.34 (1H, m), 2.17-2.24 (1H, m), 2.16 (3H, m), 1.91-2.02 (1H, m), 1.79-1.87 (1H, m), 1.68-1.76 (1H, m). The results confirm a successful synthesis of (S)-nicotine.

### Example 7

Preparation of (S)-nicotine Through a Reduction Process Using a Chiral Metal Catalyst

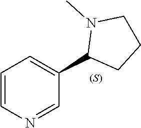

1.25 g of the Compound C prepared in Preparation Example 4-1, 50 mL of methanol, 1.0 g of triethylamine, and 35 mg of ((R)-(+)-2,2′-Bis(diphenylphosphino)-1,1′-binaphthyl)dichlororuthenium(II) as a chiral metal catalyst, were added to a 100 mL hydrogenation reactor. Hydrogen gas was then introduced into the hydrogenation reactor to maintain a pressure of 3.0 MPa, and the temperature was controlled at 90° C. When the reaction was completed, the solvent was removed through concentration, and the residue was slurried in n-heptane, filtered, and concentrated to obtain (S)-nicotine, with a purity of 59% and an optical purity of 66%.

The (S)-nicotine obtained was purified by column chromatography and characterized using nuclear magnetic resonance (NMR). The NMR data are as follows: ¹H-NMR (400 MHz, CDCl₃): δ ppm 8.54 (1H, d), 8.50 (1H, dd), 7.70 (1H, dt), 7.24-7.27 (1H, m), 3.22-3.27 (1H, m), 3.08 (1H, t), 2.27-2.34 (1H, m), 2.17-2.24 (1H, m), 2.16 (3H, m), 1.91-2.02 (1H, m), 1.79-1.87 (1H, m), 1.68-1.76 (1H, m). The results confirm a successful synthesis of (S)-nicotine.

### Example 8

Preparation of (S)-nicotine Using a Biocatalytic Method

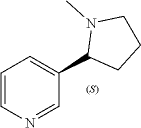

30 mg of the Compound C prepared in Preparation Example 4-2 was added to each of 21 first centrifuge tubes (5 mL each), followed by the addition of 2 mL of 0.1 M phosphate buffer. The pH of the mixture was adjusted to 6.0. Then, 32 mg of glucose was added to each first centrifuge tube and stirred until completely dissolved. Subsequently, 30 mg of the imine reductases comprising the amino acid sequences as shown in SEQ ID NO: 2-22, prepared in Preparation Example 5-2, were individually added to each first centrifuge tube. 3 mL of 0.1 M phosphate buffer, 40 mg of Enzyme 2 prepared in Preparation Example 5-1, and 40 mg of NADP salt, were added into a 5 mL second centrifuge tube, stirred until completely dissolved. Then, 0.1 mL of the solution from the second centrifuge tube was slowly added into each first centrifuge tube. The temperature was raised to 25° C., and the mixture was stirred at 300 rpm for 16 hours. 0.1 mL of the resulting solution was mixed with 0.9 mL of methanol, shaken for 1 min in, filtered, and then transferred into a 1 mL liquid-phase vial for analysis using high-performance liquid chromatography.

An area ratio of (S)-nicotine corresponds to a conversion rate, as shown in Table 2.

TABLE 2

| | Conversion | ee | |
|---|---|---|---|
| | Rate | Value | |
| Enzyme | (%) | (%) | Configuration |
| 1 | 99.5 | 99.4 | S |
| 3 | 99.6 | 100 | S |
| 4 | 94.6 | 99.1 | S |
| 5 | 89.5 | 85.5 | S |
| 6 | 88.6 | 95.3 | S |
| 7 | 0 | / | / |
| 8 | 56.4 | 88.1 | S |
| 9 | 83.2 | 99.2 | R |
| 10 | 77.9 | 87.6 | R |
| 11 | 23.5 | 99.5 | S |
| 13 | 99.5 | 99.9 | S |
| 14 | 99.5 | 99.5 | S |
| 15 | 99.8 | 99.8 | S |
| 16 | 99.5 | 99.7 | S |
| 17 | 99.5 | 99.7 | S |
| 18 | 99.8 | 99.5 | S |
| 19 | 99.5 | 99.3 | S |
| 20 | 99.3 | 99.5 | S |
| 21 | 99.5 | 99.3 | S |
| 22 | 99.9 | 99.9 | S |
| 23 | 99.3 | 99.2 | S |

Referring to the data in Table 2, it is evident that Enzymes 1, 3, 4, 13-18, and 22 exhibit superior catalytic efficiency compared to other enzymes, achieving a conversion rate exceeding 94.6%.

### Example 9

Preparation of (S)-nicotine Using a Biocatalytic Method

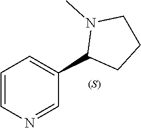

4.5 g of the Compound C prepared in Preparation Example 4-1 and 20 mL of 0.1 M phosphate buffer were added into a 50 mL three-neck round-bottom flask, the pH of the mixture was adjusted to 7.0. Then, 4.8 g of glucose was added into the three-neck round-bottom flask and stirred until completely dissolved. 10 mL of 0.1 M phosphate buffer, 0.3 g of Enzyme 1 prepared in Preparation Example 5-2, 0.04 g of Enzyme 2 prepared in Preparation Example 5-1, and 0.008 g of NADP salt were added into a 50 mL flask and stirred until fully dissolved. Next, the solution from the flask was slowly added into the three-neck round-bottom flask. The temperature was raised to 30° C., and. The resulting mixture was stirred at 300 rpm for 16 hours and filtered. The pH of the filtrate was adjusted to 10 using a sodium hydroxide solution. Then, the filtrate was extracted with methyl tert-butyl ether, dried with anhydrous sodium sulfate, and concentrated to obtain 2.6 g of (S)-nicotine, with a purity of 99%, an optical purity of 100%, and a yield of 89.5%.

The resulting (S)-nicotine was purified by column chromatography and characterized using nuclear magnetic resonance (NMR). The NMR data are as follows: ¹H-NMR (400 MHz, CDCl₃): δ ppm 8.54 (1H, d), 8.50 (1H, dd), 7.70 (1H, dt), 7.24-7.27 (1H, m), 3.22-3.27 (1H, m), 3.08 (1H, t), 2.27-2.34 (1H, m), 2.17-2.24 (1H, m), 2.16 (3H, m), 1.91-2.02 (1H, m), 1.79-1.87 (1H, m), 1.68-1.76 (1H, m). The results confirm a successful synthesis of (S)-nicotine.

### Example 10

Preparation of (S)-nicotine Using a Biocatalytic Method

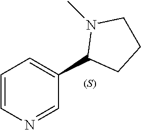

4.5 g of the Compound C prepared in Preparation Example 3-1 and 20 mL of 0.1 M phosphate buffer were added into a 50 mL three-neck round-bottom flask, the pH of the mixture was adjusted to 6.0. Then, 4.8 g of glucose was added into the three-neck round-bottom flask and stirred until completely dissolved. 10 mL of 0.1 M phosphate buffer, 0.4 g of Enzyme 3 prepared in Preparation Example 5-2, 0.04 g of Enzyme 2 prepared in Preparation Example 5-1, and 0.008 g of NADP salt were added into a 50 mL flask and stirred until fully dissolved. Next, the solution from the flask was slowly added into the three-neck round-bottom flask. The temperature was raised to 35° C., and. The resulting mixture was stirred at 300 rpm for 16 hours and filtered. The pH of the filtrate was adjusted to 10 using a sodium hydroxide solution. Then, the filtrate was extracted with ethyl acetate, dried with anhydrous sodium sulfate, and concentrated to obtain 2.4 g of (S)-nicotine, with a purity of 99%, an optical purity of 99.4%, and a yield of 82.6%.

The resulting (S)-nicotine was purified by column chromatography and characterized using nuclear magnetic resonance (NMR). The NMR data are as follows: ¹H-NMR (400 MHz, CDCl₃): δ ppm 8.54 (1H, d), 8.50 (1H, dd), 7.70 (1H, dt), 7.24-7.27 (1H, m), 3.22-3.27 (1H, m), 3.08 (1H, t), 2.27-2.34 (1H, m), 2.17-2.24 (1H, m), 2.16 (3H, m), 1.91-2.02 (1H, m), 1.79-1.87 (1H, m), 1.68-1.76 (1H, m). The results confirm a successful synthesis of (S)-nicotine.

### Example 11

Preparation of (S)-nicotine Using a Biocatalytic Method

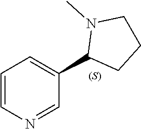

4.5 g of the Compound C prepared in Preparation Example 3-1 and 20 mL of 0.1 M phosphate buffer were added into a 50 mL three-neck round-bottom flask, the pH of the mixture was adjusted to 6.0. Then, 4.8 g of glucose was added into the three-neck round-bottom flask and stirred until completely dissolved. 10 mL of 0.1 M phosphate buffer, 0.4 g of Enzyme 12 prepared in Preparation Example 5-3, and 0.008 g of NADP salt were added into a 50 mL flask and stirred until fully dissolved. Next, the solution from the flask was slowly added into the three-neck round-bottom flask. The temperature was raised to 35° C., and. The resulting mixture was stirred at 300 rpm for 16 hours and filtered. The pH of the filtrate was adjusted to 10 using a sodium hydroxide solution. Then, the filtrate was extracted with ethyl acetate, dried with anhydrous sodium sulfate, and concentrated to obtain 2.3 g of (S)-nicotine, with a purity of 99%, an optical purity of 99.6%, and a yield of 79.2%.

The resulting (S)-nicotine was purified by column chromatography and characterized using nuclear magnetic resonance (NMR). The NMR data are as follows: ¹H-NMR (400 MHz, CDCl₃): δ ppm 8.54 (1H, d), 8.50 (1H, dd), 7.70 (1H, dt), 7.24-7.27 (1H, m), 3.22-3.27 (1H, m), 3.08 (1H, t), 2.27-2.34 (1H, m), 2.17-2.24 (1H, m), 2.16 (3H, m), 1.91-2.02 (1H, m), 1.79-1.87 (1H, m), 1.68-1.76 (1H, m). The results confirm a successful synthesis of (S)-nicotine.

It will be obvious to those skilled in the art that changes and modifications may be made, and therefore, the aim in the appended claims is to cover all such changes and modifications.

What is claimed is:

1. A method for preparing (S)-nicotine by reduction, the method comprising:
conducting a reduction process on an alkene compound shown in Formula I and/or an iminium cation compound shown in Formula II 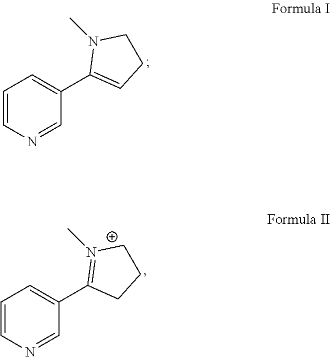
thereby producing (S)-nicotine.

2. The method of claim 1, wherein the reduction process is performed using a biocatalytic method, and the biocatalytic method comprises:
in a coenzyme cycling system, catalytically reducing the alkene compound as shown in Formula I and/or the iminium cation compound as shown in Formula II by an imine reductase, thereby producing (S)-nicotine.

3. The method of claim 2, wherein the coenzyme cycling system further comprises a coenzyme, glucose, and glucose dehydrogenase;
the coenzyme comprises NADP salt and/or NAD salt;
the glucose dehydrogenase comprises an amino acid sequence shown in SEQ ID NO: 1;
the imine reductase comprises an amino acid sequence shown in SEQ ID NOs: 2-6, SEQ ID NO: 8, SEQ ID NO: 11, SEQ ID NO: 12, or an amino acid sequence comprising at least 95% identity to the amino acid sequence of SEQ ID NO: 12;
the reduction process is carried out within a temperature range of 15 to 45° C.;
the reduction process is conducted in a buffer solution; and the buffer solution comprises phosphate buffer, Tris-HCl buffer, or TEA-HCl buffer; and
the reduction process is carried out within a pH range of 6.0 to 8.0.

4. The method of claim 1, wherein the reduction process is performed using a chiral metal catalyst, and comprises: in a hydrogen-rich atmosphere, catalytically reducing the alkene compound as shown in Formula I and/or the iminium cation compound as shown in Formula II by the chiral metal catalyst, thereby yielding (S)-nicotine.

5. The method of claim 4, wherein the chiral metal catalyst comprises a chiral iridium catalyst, a chiral ruthenium catalyst, or a chiral rhodium catalyst;
a ligand is added to the reduction process;
the reduction process is carried out within a temperature range of 50 to 100° C.;
during the reduction process, a pressure of the hydrogen gas is maintained at a range of 1.0 and 6.0 MPa; and
the reduction process is carried out within a pH range of 4.0 to 13.0.

6. The method of claim 1, wherein the alkene compound as shown in Formula I or the iminium cation compound as shown in Formula II are prepared by desalination and/or cyclization of a compound as shown in Formula III, or a salt thereof; 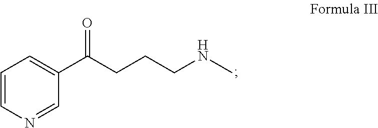
the salt of the compound of Formula III comprises hydrochloride, dihydrochloride, hydrobromide, dihydrobromide, sulfate, or bisulfate.

7. The method of claim 6, wherein a method for synthesizing the salt of the compound of Formula III comprises:
mixing a compound A with N-methylpyrrolidone and an organic base to yield a compound D; and mixing the compound D with an acid to yield the salt of the compound of Formula III; the reaction equations are as follows:

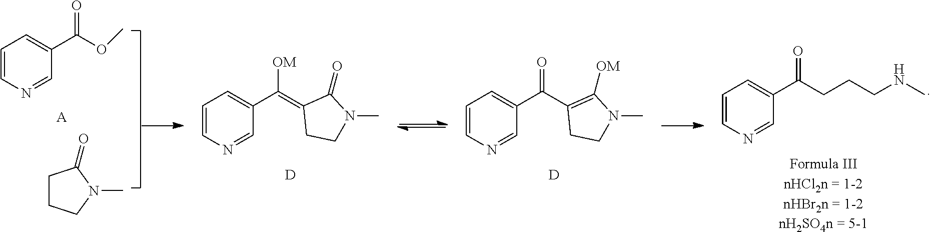

8. The method of claim 6, wherein a method for synthesizing the salt of the compound of Formula III, and the method comprises:
mixing the compound A with N-methylpyrrolidone and an organic base to form a resultant product; neutralizing the resultant product with an acid to reach a pH of 7-8, thereby yielding a compound B; and mixing the compound B with an acid to produce the salt of the compound of Formula III; the reaction equations are as follows:

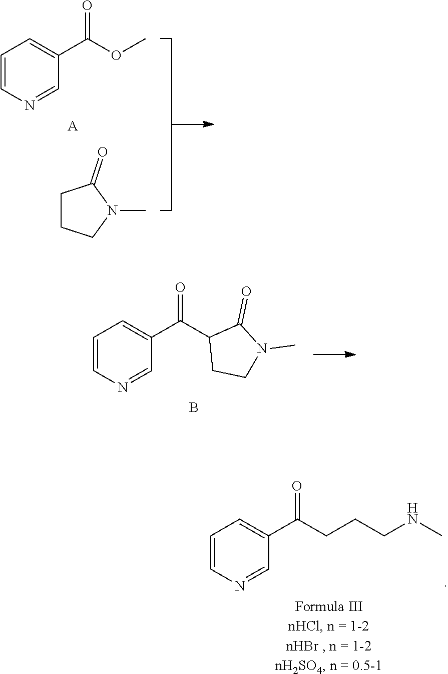

9. The method of claim 7, wherein a method for synthesizing the compound A comprises:
mixing nicotinic acid with methanol to form a mixture; and esterifying the mixture in a high acidic environment, thereby yielding the compound A.

10. The method of claim 8, wherein a method for synthesizing the compound A comprises:
mixing nicotinic acid with methanol to form a mixture; and esterifying the mixture in a high acidic environment, thereby yielding the compound A.

11. The method of claim 1, comprising: mixing nicotinic acid with methanol to form a mixture; and esterifying the mixture in a high acidic environment, thereby obtaining a compound A; mixing the compound A with N-methylpyrrolidone and the organic base to yield the compound D, or mixing the compound A with N-methylpyrrolidone and the organic base, and further adding a first acid for neutralization, to yield the compound B; mixing the compound B or the compound D with a second acid, thereby producing the salt of the compound of Formula III; desalting and cyclizing the salt of the compound of Formula III, and reducing a resulting product using a biocatalytic method or a chiral metal catalyst to obtain (S)-nicotine; the reaction equations are as follows: [chemical structure]


SEQUENCE LISTING (22 sequences)

SEQ ID NO: 1 - protein, 260 aa, synthetic construct
MYKDLEGKVVVITGSSTGLGKSMAIRFATEKAKVVVNYRSKEDEANSVLEEIKKVGGEAIAVKGDVTVESDVINLVQSAIKEFGKLDVMINNAGLENPVSSHEMSLSDWNKVIDTNLTGAFLGSREAIKYFVENDIKGTVINMSSVHEKIPWPLFVHYAASKGGMKLMTETLALEYAPKGIRVNNIGPGAINTPINAEKFADPEQRADVESMIPMGYIGEPEEIAAVAAWLASSEASYVTGITLFADGGMTQYPSFQAGR

SEQ ID NO: 2 - protein, 292 aa, synthetic construct
MRHLSVIGLGAMGSALATTLLKAGHPVTVWNRSAAKAAPLQALGATLAPSVGEAIAASDITLVCVDNYAVSQQLLDEASDAVAGKLLVQLSTGSPQGARSLESWCHTRGARYLDGAILCFPDQIGTTDASIICSGASTAFSEAEPVLRLLAPPLDHVAEAVGAAAAQDCAVAAYFAGGLLGALHGALICEVEGLPVAKVCAQFSELSPILGGDVAHLGKTLASGDFDHPYASLKTWSAAISRLAGHATDAGIDSRFPRFAADLFEEGVAQGFGQQEVSALIKVLRARNGAAQ

SEQ ID NO: 3 - protein, 292 aa, synthetic construct
MRHLSVIGLGAMGSALATTLLKAGHPVTVWNRSAAKAAPLQALGATLAPSVGAAIAASDITLVCVDNYAVSQLLLDEASDAVAGKLLVQLSTGSPQGARALESWSHARGARYLDGAILCFPAQIGTSDASIICSGASAAFSEAEPVLSLLAPTLDHVAEAVGAAAAQDCAVAAYFAGGLLGALHGALICEAEGLPVAKVCAQFSELSPILGGDVAHLGKTLASGDFDHPYASLKTWSAAISRLAGHATDAGIDSRFPRFAADLFEEGVAQGFGQQEVSALIKVLRARNGAAQ

SEQ ID NO: 4 - protein, 258 aa, synthetic construct
MRHLSVIGLGAMGSALATTLIKGGHPVTVWNRSAAKAAPLQALGATLAPSVGAAIAASDITLVCVDNYAVSQQLLDEARDAVAGKLLVQLSTGSPQGARALESWSHARGARYLDGAILCFPDQIGTSDASIICSGASAAYFAGGLLGALHGALICEAEGLPVAKVCAQFSELSPILGGDVAHLGKTLASGDFDHPYASLKTWSAAISRLAGHATDAGIDSRFPRFAADLFEEGVAQGFGQQEVSALIKVLRARNGAAL

SEQ ID NO: 5 - protein, 292 aa, synthetic construct
MRPISVIGLGAMGSALATTLLKAGHPVTVWNRSAAKATPLIALGAILAPSVSEAIAAGDITLICVDNYAVSQQLLDEASNAVTGKLVVQLSTGSPLGARTLESWCHARGACYLDGAILCFPDQIGTTDASIICSGANAAFREAEPVLRLLAPTLEHVAEAVGAAAAQDCAVAAYFAGGLLGALHGALICEAEGLPVAKVCAQFSELSPILGGDVAHLGKTLASGDFDHPYASLKTWSAAISRLTDHAADAGIDNSFPRFAADLFEEGVEQGLGQQEVSALIKVLRARNGAAQ

SEQ ID NO: 6 - protein, 292 aa, synthetic construct
MRHLSVIGLGAMGSALATTLIKAGHPVTVWNRSAAKSAPLQALGATLAPSVGAAIAASDITLVCVDNYAVSQQLLDEASDAVAGKQLVQLSTGSPQGARALESWSHARGARYLDGAILCFPDQIGTSDASIICSGANTAFSDAEPVLRLLAPTLDHVAEAVGAAAAQDCAVAAYFAGGLLGALHGALICEAEGLPVTKVCAQFSELSPILGGDVAHLGKTLASGDFDHPYASLKTWSAAISRLAGHATDAGIDSRFPRFAADLFEEGVAQGFGQQEVSALIKVLRARNGAAQ

SEQ ID NO: 7 - protein, 276 aa, synthetic construct
MGTALVEAFLAGGHATTVWNRTPGKADGVVARGAVVAETVAEAVAASPLVVVCLWDDAVVRDVLHPVADALAGRVVVNLTNGTPAQAREMAAWAAEHGVEYVDGGIMAIPPGIGTEHAFVLYSGAEAAFEAHREVLERLGAAKYLGADAGLAALFDLALLSGMYGTFAGLWHSLAMVRTENVSAAEFVPMLGPWMQAMIGGNLDRLAHQLDTGDYGHEVVSNLAMQAAAFPNIVQASLDQGIRPDLMAPIQRLMDQAVAAGHGAEDVAVVVDLLKN

SEQ ID NO: 8 - protein, 291 aa, synthetic construct
MKPTLTVIGAGRMGSALIKAFLQSGYTTTVWNRTKAKSEPLAKLGAHLADTVRDAVKRSDIIVVNVLDYDTSDQLLRQDEVTRELRGKLLVQLTSGSPALAREQETWARQHGIDYLDGAIMATPDFIGQAECALLYSGSAALFEKHRAVLNVLGGATSHVGEDVGHASALDSALLFQMWGTLFGTLQALAISRAEGIPLEKTTAFIKLTEPVTQGAVADVLTRVQQNRLTADAQTLASLEAHNVAFQHLLALCEERNIHRGVADAMYSVIREAVKAGHGKDDFAILTRFLK

SEQ ID NO: 9 - protein, 329 aa, synthetic construct
MVSSPYLNVTAYPKVRNLPWPVPGPIRVASQILELRPMTTIGFLGAGRMGSALVKSLLEAGHSVHVWNRTAEKAQALADFGAVPEPSAERAAGPAEIVIVNLLDYEASDAELRKPDVAEALKGKLLVQLTSGSPKTARETGRWAGDHGIAYLDGAIMATPNFIGGAETVILYSGSKTHFEKHEGLFKALGGKSAFVGEDFGTASALDSALLSQMWGTLFGTLQALAVCRAEGIEHDVYAGFLMSAQPMIDGAQQDLMERIRDGRDLADAQTLATVAVHNVAFHHLRDLIADRDLNPAFGDALGSLLETALRNDHQDDDFAVLARFMGAK

SEQ ID NO: 10 - protein, 293 aa, synthetic construct
MTDLGKSAVTVLGLGAMGTALAEALLAAGHPTTVWNRSPARTAGPAQRGAAVAAATAEAIAASRLIVVCLLDHTSVHAVLDGQELTGRIVVNLTSGTPGQARELDARVAERGGDHLDGAVLAVPSMIGTPDASVLYSGSRGAFDTHRPVLEVFGAADYVGADPGAASLQDAALLSAMYGQVAGVLHAFALVRSAGVTATEFLPRLVGWLTAMGGFPADAARRIDARAYADDVDAALTMQVTAVRNLVRAAREQGVSAELIAPLVPVMQRRIDDGDGGDDLAALVEVITAEEVA

SEQ ID NO: 11 - protein, 290 aa, synthetic construct
MTDKPPVTVLGLGAMGTALARTLLNAGYPTTVWNRTASKTAPLTELGAHAADSPADAIARGELVLACLLDYDSVHQTLAGTGDALRGKAFVNLTNGTPEQARALAGKLDTAYLDGGIMAVPPMIGSPGAFLFYSGEIAVFEQYRPVLESFGEAIEVGTDPGLAALHDLALLSAMYGMFGGVLQAFALTGSAGVSAASLAPLLHRWLDGMSGFIAQSAAQLDSGDFATGVVSNLAMQDTGFANLFRAAKEQGISTGQLEPLGALIRRRVEDGHGAEDLAGIVEYLKIGANA

SEQ ID NO: 12 - protein, 292 aa, synthetic construct; identical to SEQ ID NO: 3

SEQ ID NO: 13 - protein, 292 aa, synthetic construct
MRHLSVIGLGAMGSALATTLLKAGHPVTVWNRSAAKAAPLQALGATLAPSVGAAIAASDITLVCVDNYAVSQVLLDEASDAVAGKLLVQLSTGSPQGARALESWSHARGARYLDGAILCFPAQIGTSDASIICSGASAAFSEAEPVLSLLAPTLDHVAEAVGAAAAQDCAAAAYFAGGLLGALHGALICEAEGLPVAKVCAQFSELSPILGGDVAHLGKTLASGDFDHPYASLKTWSAAISRLAGHATDAGIDSRFPRFAADLFEEGVAQGFGQQEVSALIKVLRARNGAAQ

SEQ ID NO: 14 - protein, 292 aa, synthetic construct
MRHLSVIGLGAMGSALATTLLKAGHPVTVWNRSAAKAAPLQALGATLAPSVGAAIAASDITLVCVDNYAVSQQLLDEASDAVAGKLLVQLSTGSPQGARALESWSHARGARYLDGAILCFPAQIGTSDASIICSGASAAFSEAEPVLRLLAPTLDHVAEAVGAAAAQDCAVAAYFAGGLLGALHGALICEAEGLPVAKVCAQFSELSPILGGDVAHLGKTLASGDFDHPYASLKTWSAAISRLAGHATDAGIDSRFPRFAADLFEEGVAQGFGQQEVSALIKVLRARNGAAQ

SEQ ID NO: 15 - protein, 292 aa, synthetic construct
MRHLSVIGLGAMGSALATTLLKAGHPVTVWNRSAAKAAPLQALGATLAPSVGAAIAASDITLVCVDNYAVSQQLLDEASDAVAGKLLVQLSTGSPQGARALESWSHARGARYLDGAILCFPAQIGTSDASIICSGASAAFSEAEPVLRLLAPTLDHVAEAVGAAAAQDCAVAAYFAGGLLGALHGALICEAEGLPVAKVCAQFSELSPILGGDVAHLGKTLASGDFDHPGASLKTWSAAISRLAGHATDAGIDSRFPRFAADLFEEGVAQGFGQQEVSALIKVLRARNGAAQ

SEQ ID NO: 16 - protein, 292 aa, synthetic construct
MRHLSVIGLGAMGSALATTLLKAGHPVTVWNRSAAKAAPLQALGATLAPSVGAAIARSDITLVCVDNYAVSQLLLDEASDAVAGKLLVQLSTGSPQGARALESWSHARGARYLDGAILCFPAQIGTSDASIICSGASAAFSEAEPVLSLLAPTLDHVAEAVGAAAAQDCAAVAYFGGGLLGALHGALICEAEGLPVAKVCAQFSELSPILGGDVAHLGKTLASGDFDHPTASLKTWSAAIGRLAGHATDAGIDSRFPRFAADLFEEGVAQGFGQQEVSALIKVLRARNGAAQ

SEQ ID NO: 17 - protein, 292 aa, synthetic construct
MRHLSVIGLGAMGSALATTLLKAGHPVTVWNRSAAKAAPLQALGATLAPSVGAAIARSDITLVCVDNYAVSQLLLDEASDAVAGKLLVQLSTGSPQGARALESWSHARGARYLDGAILCFPAQIGTSDASIICSGASAAFSEAEPVLSLLAPTLDHVAEAVGAAAAQDCAAVAYFGGGLLGALHGALICEAEGLPVAKVCAQFSELSPILGGDVAHLGKTLASGDFDHPYASLKTWSAAIGRLAGHATDAGIDSRFPRFAADLFEEGVAQGFGQQEVSALIKVLRARNGAAQ

SEQ ID NO: 18 - protein, 292 aa, synthetic construct
MRHLSVIGLGAMGSALATTLLKAGHPVTVWNRSAAKAAPLQALGATLAPSVGAAIARSDITLVCVDNYAVSQLLLDEASDAVAGKLLVQLSTGSPQGARALESWSHARGARYLDGAILCFPAQIGTSDASIICSGASAAFSEAEPVLSLLAPTLDHVAEAVGAAAAQDCAAAAYFGGGLLGALHGALICEAEGLPVAKVCAQFSELSPILGGDVAHLGKTLASGDFDHPYASLKTWSAAIGRLAGHATDAGIDSRFPRFAADLFEEGVAQGFGQQEVSALIKVLRARNGAAQ

SEQ ID NO: 19 - protein, 292 aa, synthetic construct
MRHLSVIGLGAMGSALATTLLKAGHPVTVWNRSAAKAAPLQALGATLAPSVGAAIARSDITLVCVDNYAVSQLLLDEASDAVAGKLLVQLSTGSPQGARALESWSHARGARYLDGAILCFPAQIGTSDASIICSGASAAFSEAEPVLSLLAPTLDHVAEAVGAAAAQDCAVVAYFGGGLLGALHGALICEAEGLPVAKVCAQFSELSPILGGDVAHLGKTLASGDFDHPYASLKTWSAAIGRLAGHATDAGIDSRFPRFAADLFEEGVAQGFGQQEVSALIKVLRARNGAAQ

SEQ ID NO: 20 - protein, 292 aa, synthetic construct
MRHLSVIGLGAMGSALATTLLKAGHPVTVWNRSAAKAAPLQALGATLAPSVGAAIARSDITLVCVDNYAVSQQLLDEASDAVAGKLLVQLSTGSPQGARALESWSHARGARYLDGAILCFPAQIGTSDASIICSGASAAFSEAEPVLRLLAPTLDHVAEAVGAAAAQDCAVAAYFGGGLLGALHGALICEAEGLPVAKVCAQFSELSPILGGDVAHLGKTLASGDFDHPYASLKTWSAAISRLAGHATDAGIDSRFPRFAADLFEEGVAQGFGQQEVSALIKVLRARNGAAQ

SEQ ID NO: 21 - protein, 292 aa, synthetic construct
MRHLSVIGLGAMGSALATTLLKAGHPVTVWNRSAAKAAPLQALGATLAPSVGAAIARSDITLVCVDNYAVSQQLLDEASDAVAGKLLVQLSTGSPQGARALESWSHARGARYLDGAILCFPAQIGTSDASIICSGASAAFSEAEPVLRLLAPTLDHVAEAVGAAAAQDCAVAAYFGGGLLGALHGALICEAEGLPVAKVCAQFSELSPILGGDVAHLGKTLASGDFDHPYASLKTWSAAIARLAGHATDAGIDSRFPRFAADLFEEGVAQGFGQQEVSALIKVLRARNGAAQ

SEQ ID NO: 22 - protein, 292 aa, synthetic construct
MRHLSVIGLGAMGSALATTLLKAGHPVTVWNRSAAKAAPLQALGATLAPSVGAAIARSDITLVCVDNYAVSQQLLDEASDAVAGKLLVQLSTGSPQGARALESWSHARGARYLDGAILCFPAQIGTSDASIICSGASAAFSEAEPVLRLLAPTLDHVAEAVGAAAAQDCAVAAYFGGGLLGALHGALICEAEGLPVAKVCAQFSELSPILGGDVAHLGKTLASGDFDHPTASLKTWSAAISRLAGHATDAGIDSRFPRFAADLFEEGVAQGFGQQEVSALIKVLRARNGAAQ